SEQUENCE LISTING (22 sequences)

SEQ ID NO: 1 - DNA, 97 bp, Artificial Sequence
  Description of Artificial Sequence Synthetic oligonucleotide
tgctgttgacagtgagcgagccaaggtaccttacaccaaatagtgaagccacagatgtatttggtgtaaggtaccttggcctgcctactgcctcgga

SEQ ID NO: 2 - DNA, 19 bp, Artificial Sequence
  Description of Artificial Sequence Synthetic oligonucleotide
ccaaggtaccttacaccaa

SEQ ID NO: 3 - DNA, 97 bp, Artificial Sequence
  Description of Artificial Sequence Synthetic oligonucleotide
tgctgttgacagtgagcgcggacttcttggatttggaatttagtgaagccacagatgtaaattccaaatccaagaagtccttgcctactgcctcgga

SEQ ID NO: 4 - DNA, 19 bp, Artificial Sequence
  Description of Artificial Sequence Synthetic oligonucleotide
gacttcttggatttggaat

SEQ ID NO: 5 - DNA, 97 bp, Artificial Sequence
  Description of Artificial Sequence Synthetic oligonucleotide
tgctgttgacagtgagcgcgctctagccctcttaattctatagtgaagccacagatgtatagaattaagagggctagagcatgcctactgcctcgga

SEQ ID NO: 6 - DNA, 19 bp, Artificial Sequence
  Description of Artificial Sequence Synthetic oligonucleotide
ctctagccctcttaattct

SEQ ID NO: 7 - DNA, 97 bp, Artificial Sequence
  Description of Artificial Sequence Synthetic oligonucleotide
tgctgttgacagtgagcgcggaaattgactccacgttatatagtgaagccacagatgtatataacgtggagtcaatttccttgcctactgcctcgga

SEQ ID NO: 8 - DNA, 19 bp, Artificial Sequence
  Description of Artificial Sequence Synthetic oligonucleotide
gaaattgactccacgttat

SEQ ID NO: 9 - DNA, 97 bp, Artificial Sequence
  Description of Artificial Sequence Synthetic oligonucleotide
tgctgttgacagtgagcgaccgtattagaacattctctaatagtgaagccacagatgtattagagaatgttctaatacgggtgcctactgcctcgga

SEQ ID NO: 10 - DNA, 19 bp, Artificial Sequence
  Description of Artificial Sequence Synthetic oligonucleotide
cgtattagaacattctcta

SEQ ID NO: 11 - DNA, 97 bp, Artificial Sequence
  Description of Artificial Sequence Synthetic oligonucleotide
tgctgttgacagtgagcgacctgcgttatacagagatatatagtgaagccacagatgtatatatctctgtataacgcagggtgcctactgcctcgga

SEQ ID NO: 12 - DNA, 19 bp, Artificial Sequence
  Description of Artificial Sequence Synthetic oligonucleotide
ctgcgttatacagagatat

SEQ ID NO: 13 - DNA, 97 bp, Artificial Sequence
  Description of Artificial Sequence Synthetic oligonucleotide
tgctgttgacagtgagcgcgcactaatccggaagaacaaatagtgaagccacagatgtatttgttcttccggattagtgcttgcctactgcctcgga

SEQ ID NO: 14 - DNA, 19 bp, Artificial Sequence
  Description of Artificial Sequence Synthetic oligonucleotide
cactaatccggaagaacaa

SEQ ID NO: 15 - DNA, 97 bp, Artificial Sequence
  Description of Artificial Sequence Synthetic oligonucleotide
tgctgttgacagtgagcgcgctttgcaagttctgagaatatagtgaagccacagatgtatattctcagaacttgcaaagcttgcctactgcctcgga

SEQ ID NO: 16 - DNA, 19 bp, Artificial Sequence
  Description of Artificial Sequence Synthetic oligonucleotide
ctttgcaagttctgagaat

SEQ ID NO: 17 - DNA, 97 bp, Artificial Sequence
  Description of Artificial Sequence Synthetic oligonucleotide
tgctgttgacagtgagcgcgcaccctgactctcactaaattagtgaagccacagatgtaatttagtgagagtcagggtgcttgcctactgcctcgga

SEQ ID NO: 18 - DNA, 19 bp, Artificial Sequence
  Description of Artificial Sequence Synthetic oligonucleotide
caccctgactctcactaaa

SEQ ID NO: 19 - DNA, 97 bp, Artificial Sequence
  Description of Artificial Sequence Synthetic oligonucleotide
tgctgttgacagtgagcgcggagatgcaatgaacatagaatagtgaagccacagatgtattctatgttcattgcatctccttgcctactgcctcgga

SEQ ID NO: 20 - DNA, 19 bp, Artificial Sequence
  Description of Artificial Sequence Synthetic oligonucleotide
gagatgcaatgaacataga

SEQ ID NO: 21 - DNA, 97 bp, Artificial Sequence
  Description of Artificial Sequence Synthetic oligonucleotide
tgctgttgacagtgagcgagcagacagattcctttgttaatagtgaagccacagatgtattaacaaaggaatctgtctgcgtgcctactgcctcgga

SEQ ID NO: 22 - DNA, 19 bp, Artificial Sequence
  Description of Artificial Sequence Synthetic oligonucleotide
cagacagattcctttgtta

(19) United States
(12) Patent Application Publication
(10) Pub. No.: US 2011/0035814 A1
(43) Pub. Date: Feb. 10, 2011

(54) COOPERATING ONCOGENES IN CANCER

(75) Inventors: Lars Zender, Hannover (DE); Scott W. Lowe, Cold Spring Harbor, NY (US); Mona S. Spector, Cold Spring Harbor, NY (US); Wen Xue, Cold Spring Harbor, NY (US)

Correspondence Address: WilmerHale/Cold Spring Harbor Laboratory, 399 Park Avenue, New York, NY, 10022, US

(73) Assignee: COLD SPRING HARBOR LABORATORY, Cold Spring Harbor, NY (US)

(21) Appl. No.: 12/301,980

(22) PCT Filed: May 23, 2007
(86) PCT No.: PCT/US2007/012592

§ 371 Date: Oct. 27, 2010

Related U.S. Application Data
(60) Provisional application No. 60/802,900, filed on May 23, 2006.

(51) Int. Cl. C12Q 1/68; C12N 5/071; A01K 67/027; C12Q 1/02

(52) U.S. Cl. 800/3; 435/6; 435/375; 800/18; 435/325; 435/29

(57) ABSTRACT

This invention provides methods of diagnosis, drug screening, and treatment based on the discovery that cIAP1 and Yap are co-amplified oncogenes that cooperate to contribute to oncogenesis and tumor maintenance.

This invention was supported in part by the United States Government under National Institutes of Health Grants CA 13106, CA87497, and CA1053388. The Government may have certain rights in this invention.

### BACKGROUND OF THE INVENTION

Cancer is the second leading cause of death in industrial countries. More than 70% of all cancer deaths are due to carcinomas, i.e., cancers of epithelial organs. Most carcinoma tumors show initial or compulsory chemoresistance. This property makes it very difficult to cure these tumors when they are detected in progressed stages. Primary forms of liver cancers include hepatocellular carcinoma, biliary tract cancer and hepatoblastoma. Hepatocellular carcinoma is the fifth most common cancer worldwide but, owing to the lack of effective treatment options, constitutes the leading cause of cancer deaths in Asia and Africa and the third leading cause of cancer death worldwide. Parkin et al., “Estimating the world cancer burden: Globocan 2000,” Int. J. Cancer 94, 153-156 (2001).

The risk factors for liver cancer include excessive alcohol intake or other toxins, such as iron, aflatoxin B1 and also the presence of other infections such as hepatitis B and C. Alison & Lovell, “Liver cancer: the role of stem cells,” Cell Prolif. 38, 407-421 (2005). The only curative treatments for hepatocellular carcinoma are surgical resection or liver transplantation, but most patients present with advanced disease and' are not candidates for surgery. To date, systemic chemotherapeutic treatment is ineffective against hepatocellular carcinoma, and no single drug or drug combination prolongs survival. Llovet et al., “Hepatocellular carcinoma,” Lancet 362, 1907-1917 (2003). However, despite its clinical significance, liver cancer is understudied relative to other major cancers.

One of the difficulties in identifying appropriate therapeutics for tumor cells in vivo is the limited availability of appropriate test material. Human tumor lines grown as xenographs are unphysiological, and the wide variation between human individuals, not to mention treatment protocols, makes clinical studies difficult. Consequently, oncologists are often forced to perform correlative studies with a limited number of highly dissimilar samples, which can lead to confusing and unhelpful results.

Non-human animal models provide a useful alternative to studies in humans and to human tumor cell lines grown as xenographs, as large numbers of genetically-identical individuals can be treated with identical regimens. Moreover, the ability to introduce germline mutations that affect oncogenesis into these animals increases the power of the models.

To investigate the basic mechanisms of carcinogenesis and to test new potential cancer agents and therapies, however, realistic carcinoma non-human animal models are urgently needed. So far there have been two major ways to create carcinoma non-human animal models: (i) the generation of transgenic or chimeric non-human animals that express oncogenes under the control of a tissue specific promoter and (ii) carcinomas that were induced by chemical carcinogens. Both approaches have several disadvantages.

Current animal models for cancer are based largely on classical transgenic approaches that direct expression of a particular oncogene to an organ of choice using a tissue specific promoter. See, e.g., Wang et al., “Activation of the Met receptor by cell attachment induces and sustains hepatocellular carcinomas in transgenic mice,” J Cell Biol. 153, 1023-1034 (2001). Although such models have provided important insights into the pathogenesis of cancer, they express the active oncogene throughout the entire organ, a situation that does not mimic spontaneous tumorigenesis. Moreover, incorporation of additional lesions, such as a second oncogene or loss of a tumor suppressor, requires genetic crosses that are time consuming and expensive, and again produce whole tissues that are genetically altered. Finally, traditional transgenic and knockout strategies do not specifically target liver progenitor cells, which may be the relevant initiators of the disease.

Cancer therapies that directly target oncogenes are based on the premise that cancer cells require continuous oncogenic signaling for survival and proliferation. Non-human animal models expressing oncogenes in genetic backgrounds that lack, or have down-regulated, tumor suppressor genes can thus serve as valuable tools to study tumor initiation, maintenance, progression, treatment and regression. However, responses to the targeting drugs are often heterogeneous, and chemoresistance and other resistance is a problem. Because most anticancer agents were discovered through empirical screens, efforts to overcome resistance are hindered by a limited understanding of why these agents are effective and when and how they become less effective or ineffective.

Variations in both non-human animal strains and promoters used to drive expression of oncogenes complicate the interpretation of cancer mechanistics and treatment analyses. First, intercrossing strategies to obtain non-human animals of the desired genetic constellation are extremely time consuming and costly. Second, the use of certain cell-selective promoters can result in a cell-bias for tumor initiation. For example, the mouse mammary tumor virus (MMTV) promoter and the Whey Acidic Protein (WAP) promoter are commonly used to model breast cancer development in mice, and yet may not target all subtypes of mammary epithelia, i.e., stem cell and non-stem cells.

An additional difficulty in identifying and evaluating the efficacy of cancer agents on tumor cells and understanding the molecular mechanisms of the cancers and their treatment in the current non-human animal models in vivo is the limited availability of appropriate material. A homogenous expression of the respective oncogene in all epithelial cells of an organ creates an unphysiological condition, as tumors are known to originate within genetic-mosaics. It is therefore important to use a valid model to target distinct genetic pathways and to identify new therapeutics for the treatment of cancers such as liver cancer.

### SUMMARY OF THE INVENTION

The invention provides in vivo and in vitro systems and methods for the study of the effects of tumorigenesis, tumor maintenance, tumor regression and altered expression of a gene activity, on the descendants of embryonic liver progenitor cells, or primary hepatocytes, that have been engineered to produce hepatocellular carcinomas.

The liver cancer model of this invention is made by altering hepatocytes to increase oncogene expression, to reduce tumor suppressor gene expression or both and by transplanting the resulting hepatocytes into a recipient non-human animal. The spontaneous mutations arising in tumors initiated by different oncogenic lesions are compared to alterations observed in human cancers. Preferably, the transplanting is carried out so that the hepatocytes engraft the liver of the animal and a liver cancer tumor develops from at least one of the altered hepatocytes. Alternatively, the altered hepatocytes are transplanted subcutaneously into a non-human animal so as to develop a tumor.

The non-human animal model of hepatocellular carcinoma embodied herein is useful for identifying molecular targets for drug screening, for identifying interacting gene activities, for identifying therapeutic treatments and for identifying candidates for new therapeutic treatments. The invention also provides methods and non-human animals produced by the methods that are useful for understanding liver cancer and its treatments, and in particular, for identifying and studying inhibitors and activators associated with liver tumor cell growth and growth inhibition, cell death through apoptotic pathways, and changes in apoptotic pathway components that affect drug sensitivity and resistance in tumorigenic cells.

The genetically tractable, transplantable in situ liver cancer model of this invention is characterized by genetically defined hepatocellular carcinomas that are preferably traceable by external green fluorescent protein (GFP) imaging. To further characterize the genetic defects in these tumors, genomic profiling, e.g., representational oligonucleotide microarray analysis (ROMA), can be used to scan the carcinomas for spontaneous gains and losses in gene copy number. Detecting genomic copy number changes through such high resolution techniques can be useful to identify oncogenes (amplifications or gains) or tumor suppressor genes (deletions or losses). Identification of overlapping genomic regions altered in both human and mouse gene array datasets may further aid in pinpointing of regions of interest that can be further characterized for alterations in RNA and protein expression to identify candidates are most likely to contribute to the disease phenotype and to be the “driver gene” for amplification.

Using “forward genetics” in combination with gene profiling (e.g., ROMA) and the non-human animal models of this invention, important insights into the molecular mechanisms of hepatocarcinogenesis, growth, maintenance, regression and remission can be obtained. The models of the invention can directly evaluate the potency of various oncogenes in producing anti-apoptotic phenotypes, and various tumor suppressor genes in producing apoptotic phenotypes. Candidate oncogenes or tumor suppressors can be rapidly validated in the mouse model of the invention by overexpression, or by using stable RNAi technology, respectively. The invention is also useful in analyzing and evaluating genetic constellations that confer chemoresistance or poor prognosis. Furthermore, the invention is useful for identifying and evaluating new therapies for the treatment of carcinomas.

In some embodiments, this invention provides a method of diagnosing liver cancer in a human patient, comprising detecting in the liver DNA sample of a patient the amplification of a nucleic acid sequence (e.g., cIAP1, cIAP2 or Yap) in chromosomal region 11q22, wherein said amplification indicates that the patient has, or is susceptible of developing, liver cancer.

This invention also provides a method of selecting a cancer patient for treatment with an inhibitor of IAP, CDK, RAF, or MEK, comprising detecting in a cancer DNA sample of the patient amplification of a nucleic acid sequence in chromosomal region 11q22, wherein said amplification indicates that the patient will be responsive to said treatment.

This invention also provides a method of inhibiting the growth of a cancer cell in vivo or in vitro (e.g., a human cancer cell such as an epithelial cancer cell, including a liver cancer cell), comprising contacting the cell with a Yap inhibitor, such as a small molecule, an interfering RNA, an antisense molecule, an antibody, or a peptide mimetic that inhibits the expression of Yap.

This invention further provides a method of identifying a molecule for treating cancer, comprising: providing a sample comprising Yap, contacting said sample with a candidate molecule, and detecting, in said sample, a decrease in the activity of said Yap, wherein said decrease indicates that the candidate molecule is useful for treating cancer. This method includes cell-based assays, using a mammalian cell such as a mouse cell or a human cell, and cell-free assays.

This invention also provides a mouse at least some of whose cells comprise a genome comprising a heterologous nucleic acid sequence comprising a Yap-coding sequence and an expression control sequence linked operatively thereto, wherein the mouse has cancer, or is more susceptible of developing cancer as compared to a control mouse not having said heterologous nucleic acid sequence. In those cells, the Yap sequence is overexpressed (expressed at a level higher than the endogenous Yap level). Any expression control sequence well known in the art can be used, including tissue-specific promoter sequence, viral LTR sequences, inducible promoter sequences, and the like. The mouse can be a transgenic mouse, or a chimeric mouse some or all of whose hepatocytes comprise said genome. In some embodiments, the mouse cells further comprise a second heterologous nucleic acid sequence comprising a cIAP1-coding sequence and a second expression control sequence linked operatively thereto. In these mice, the Yap and cIAP-1 codings sequences are co-expressed in the same cell, and this co-expression makes the cells prone to cancerous transformation. In some embodiments, these cells further comprise additional activated or overexpressed oncogenes (e.g., ras, raf, myc, and Akt), and/or a reduced function (e.g., due to null mutation or RNA interference) of a tumor suppressor gene (e.g., p53, PTEN, Rb, p16 or p19). The mice of the invention can be used to identify anti-cancer therapeutics. An anti-cancer therapeutic can inhibit the growth, including causing regression, of a cancer developed in the animal.

Mammalian cells having the same genetic features as these mice are also useful, for example, for drug screening assays.

This invention provides cancer treatment methods in which both Yap and cIAP1 are targeted for inhibition. Inhibitors of Yap and cIAP1 expression or function, such as RNA interference molecules, antisense molecules, small molecules, antibodies, peptides, peptide mimetics, can all be used.

In some embodiments, inhibitors of Yap or cIAP1 can be used in conjunction with conventional chemotherapy agents or with targeted therapy agents. For example, a Yap or cIAP1 inhibitor can be used with a MEK, CDK, EGFR, BRAF, or RAF inhibitor. In preferred embodiments, the patients for such treatments have been pre-selected for displaying amplification in the 11q22 chromosomal region.

### BRIEF DESCRIPTION OF THE FIGURES

FIG. 1: Development and characterization of a new orthotopic, genetically tractable mouse model for hepatocellular carcinoma. (a) Schematic outline of two embodiments of producing a non-human animal liver cancer model of this invention. E-Cadherin+mouse hepatoblasts are isolated from day 13-15 mouse liver using the MACS® indirect labeling system in combination with the ECCD-1 E-Cadherin antibody. Purified hepatoblasts are grown in short term primary-culture on irradiated NIH-3T3 feeder layers. The hepatoblasts are infected with GFP-tagged murine stem cell virus (MSCV) based retroviruses expressing oncogenes of interest (e.g., the H-ras oncogene) and/or expression cassettes for short hairpin RNAs directed against tumor suppressor genes (e.g., p53). After viral transduction, infected hepatoblasts are either injected into the spleens of retrorsine-conditioned recipient mice or subcutaneously into NCR nu/nu mice. Retrorsine efficiently blocks the cell cycle of hepatocytes and additionally causes a moderate liver damage by triggering apoptosis in a small number of hepatoblasts. Using this approach, after intrasplenic transplantation, genetically modified hepatocytes migrate via the portal vein into the recipient liver and engraft the organ. Transplanted hepatoblasts harboring the defined genetic lesions clonally expand and hepatocellular carcinomas develop in the liver. Tumor onset and growth kinetics can be monitored by external whole body GFP-imaging as all viral vectors carry a GFP expression cassette. (b) Kaplan-Meier curve for survival times of mice transduced with different oncogenes (myc, akt, H-rasV12). All groups succumb to death much earlier than mice injected with p53-I- control vector alone.

FIG. 2: Genome-wide analysis of copy number alterations in mouse hepatocellular carcinoma (HCC). DNA from tumors and subjected to 85K ROMA. Plotted is the normalized log-ratio for each oligo probe and ordered according to genome position, derived from the May 2004 freeze of the draft mouse genome sequence (http://www.genome.ucsc.edu). (a) Representative profiles of 3 mouse HCCs. HCC-7 and HCC-9, both derived from p53−/−; c-myc hepatoblasts, contained an amplification on chromosome 9. HCC-11, derived from p53−/−; Akt hepatoblasts, did not. (b) Expanded view of chromosome 9 reveals a 1.9 Mb amplicon (HCC-9) and a 1.2 Mb (HCC-7) amplicon containing the c-IAP-1 and c-IAP-2 genes. (c) Quantitative PCR with primers specific for the c-IAP-1 gene revealed higher copy numbers for 2 additional p53−/−; c-myc HCCs (HCC-13 and HCC-14), while non-c-myc tumors (HCC-15 and HCC-17) have a normal IAP copy number. (d) Summary of c-IAP-1/2 amplification relative to genetic background. (e) c-IAP-1 and c-IAP-2 mRNA levels are elevated in tumors containing the amplicon. Levels of IAP RNA relative to actin were determined by quantitative RT-PCR and normalized to normal liver.

FIG. 3: Genome-wide analysis of a human hepatocellular carcinoma analyzed with 36K ROMA. (a) The three peaks indicate amplicons containing the MET-oncogene, Cyclin D and c-IAP1/2 (left to right). (b) Expanded view of chromosome 11 showing the amplicons containing cyclin D and c-IAP1/2. (c) 1/25 human HCCs have elevated c-IAP-1 and c-IAP-2 gene copy numbers as determined by quantitative PCR off genomic DNA. (d) c-IAP-1 and/or c-IAP-2 mRNA levels are elevated in 4/25 HCCs as determined by quantitative RT-PCR.

FIG. 4: c-IAP-1 overexpression accelerates tumor growth. E-Cadherin+hepatoblasts were either double-infected with c-myc+control vector or c-myc+myc-tag-c-IAP-1. 10×106 cells were subcutaneously injected into irradiated NCR nu/nu mice. (a) Overexpression of c-IAP-1 in primary liver cells was confirmed by western blot analysis using an a -myc-tag antibody. In addition, four out of six c-myc+c-IAP-1 double infected tumors show accelerated tumor growth compared to c-myc+vector. Tumor size was assessed by caliper measurement of subcutaneously growing tumors. (b) All tumors showed accelerated growth contain the c-IAP-1 provirus as assayed by PCR. All analyzed tumors contained the c-myc-provirus DNA.

FIG. 5: Suppression of c-IAP-1 in HCC cells slows tumor growth. (a) Schema for testing knock-down of c-IAP-1 expression in vivo. Cells from tumors containing the c-IAP1/2 amplicon are outgrown briefly and infected either with a retrovirus expressing a short hairpin (miR30 design) RNA directed against c-IAP-1 or with control vector. After puromycin-selection, cells were injected subcutaneously into NCR nu/nu mice. (b) One out of four short hairpins directed against c-IAP-1 suppresses c-IAP-1 expression. NIH 3T3 cells were transiently transfected with pcDNA-myc tag-c-IAP-1 together with the respective hairpin. Western blot was performed using an α-myc-tag antibody. c-IAP-1- hairpin “1477” shows>95% knockdown (c) Tumors with stable RNAi mediated knockdown of c-IAP-1 show decelerated tumor growth compared to control vector infected tumors. Growth of subcutaneous tumors was assesed by caliper measurement.

FIG. 6: Influence of c-IAP-1 overexpression on proliferation and apoptosis in cultured hepatoblasts. (a) ECadherin+hepatoblasts were infected with a neomycin selectable retrovirus overexpressing c-myc and a puromycin selectable retrovirus overexpressing c-IAP-1 or control vector. After neomycin/puromycin selection cells were plated at 4.5×103 cells/cm2 and growth rate was assessed by daily counting of the total cell number. c-IAP-1 overexpressing cells have a slight growth advantage. (b) c-myc+c-IAP-1 or c-myc+vector double infected.

FIG. 7: An example of subcutaneous liver cancer model of this invention on the genetic constellation p53−/−+Akt-over expression and its uses in evaluating tumor therapy. Akt is an apoptotic regulator that is activated in many cancers and may promote drug resistance in vitro (Mayo et al., “PTEN protects p53 from Mdm2 and sensitizes cancer cells to chemotherapy,” J. Biol. Chem. 277: 5484-5489 (2002)). The graph shows that the tumor's intrinsic chemoresistance against the cancer drug Gemcitabine® (brand name “Gemzar” in the Figure) can be reversed by application of a downstream effector of Akt, the mTOR (mammalian target of rapamycin) inhibitor Rapamycin:

FIG. 8: cIAP1 enhances the tumorigenicity of Myc overexpressing p53−/−hepatoblasts. (a) p53−/−hepatoblasts were double-infected with myc plus myc-tagged-cIAP1 or myc plus vector and were subcutaneously injected into the rear flanks of nude mice (n=6 for each group). Tumor size was assesed by caliper measurement. Shown is a representative of three independent experiments. (b) cIAP1 does not enhance the tumorigenicity of H-rasV12 overexpressing p53-I- hepatoblasts. n=6 for each group. (c) cIAP1 does not enhance the tumorigenicity of Akt overexpressing p53−/−hepatoblasts.

FIG. 9. Tumors bearing the 9qA1 amplicon show delayed growth upon cIAP1 and cIAP2 suppression. (a) Stable suppression of cIAP1 and cIAP2 slows tumor growth of p53−/−; myc mouse hepatoma cells that contain the 9qA1 amplicon. Tumorigenicity of the cells described in (a) after injection into the rear flanks of nude mice. Growth of subcutaneous tumors was assessed by caliper measurement. (b) Stable suppression of cIAP1 and cIAP2 does not slow tumor growth of p53−/−; myc mouse hepatoma cells that do not contain the 9qA1 amplicon. (c) Stable suppression of p53 does not slow tumor growth of p53−/−; myc mouse hepatoma cells that contain the 9qA1 amplicon.

FIG. 10 is a panel of graphs showing that Yap confers a proliferative advantage, has oncogenic properties and is required for liver tumor progression. (a) The proliferation rates of p53−/−; myc hepatoblasts expressing Yap or a control vector were assessed by the fraction of nuclei incorporating BrdU after a 1 hour pulse. (b-d) The tumorigenicity of p53−/−liver progenitor cells co-expressing the indicated oncogene (upper left) with a control vector or Yap was assessed by caliper measurement following subcutaneous injection into the rear flanks of nude mice (>n=4 per group). (e) The tumorigenicity of 9qA1 positive cells infected with retroviral vectors expressing short hairpin RNAs (shRNAs) targeting Yap (sh Yap 1 or sh Yap 2) or control vector (n=6 per group).

FIG. 11 is a graph demonstrating that cIAP1 and Yap synergize to drive tumorigenesis. As described in Example 11, p53−/−; myc liver progenitor cells were infected with Yap, cIAP1 or control vector or co-infected with Yap+cIAP1 and then transplanted subcutaneously into nude mice. n=6 for each group.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The methods and techniques of the present invention are generally performed according to conventional methods well known in the art and as described in various general and more specific references that are cited and discussed throughout the present specification, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2003); Harlow and Lane, Antibodies: A Laboratory Manual, Cold Spring Harbor Laboratory Press, Cold Spring Harbor, N.Y. (1990); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All of the above and any other publications, patents and published patent applications referred to in this application are specifically incorporated by reference herein.

The genetically tractable, transplantable in situ liver or hepatocellular cancer model of the invention offers unique advantages. This invention employs the proliferative capacity of the liver to enable the altered hepatocytes to reconstitute liver tissue. Large amounts of primary epithelial cells can be isolated according to standardized protocols either from adult mouse livers or from embryonic mouse livers. The primary culture conditions for embryonic, as well as adult primary hepatocytes, are based on well-established protocols and are less complex compared to other epithelial primary cultures. A sample of the primary cells can be used for RT-PCR characterization for liver specific markers to rule out overgrowing by non-parenchymal cells.

Primary adult or embryonic hepatocyte cultures can be genetically modified by infection with lentiviral- or retroviral vectors carrying various genetic alterations, including oncogenes or short hairpin RNAs against tumor suppressor genes. Virally transduced primary hepatocytes can efficiently engraft the livers of non-human animals after transplantation into their portal vein or spleen. In the case of certain genetic configurations, mice developed hepatocellular carcinomas that could be visualized by whole body fluorescence imaging. For example, introduction of a myc retrovirus into p53 deficient hepatocytes produced highly aggressive tumors that show many features of human hepatocellular carcinoma. Overall, it provides rapid generation of genetically defined hepatocellular carcinomas.

The invention embodies a method of making a non-human animal bearing a liver cancer using transplanted hepatocytes altered to increase oncogene expression, to reduce tumor suppressor gene expression or both. Preferably, the hepatocytes are virally transduced with a vector expressing an oncogene or a short hairpin RNA against a tumor suppressor gene and subsequently transplanted into a recipient non-human animal wherein the animal develops liver tumors from at least one of the hepatocytes with altered gene expression.

This model has several features that make it suitable for studying the biology of liver cancer and other epithelial malignancies. First, the ability to manipulate liver progenitor cells ex vivo allows the rapid production and analysis of tumors with complex genotypes without the cost and effort associated with genetic intercrossing of cancer prone strains. For example, one can use a combination of knockout target cells and retroviral transduction to introduce multiple (e.g., two or three) defined oncogenic lesions and a reporter into liver progenitor cells, allowing quick validation of oncogenes in vivo. Second, the fact that the system relies on transplantation of progenitor cells implies that the recipient non-human animals can also have different genotypes, thereby facilitating studies of tumor-host interactions. This feature will also facilitate forward genetic screens for genes or shRNAs that affect the biology of liver and other epithelial (e.g., breast) cancer. Third, the model can use bipotential tissue progenitors as the cancer-initiating cell, making it suitable for studying a potential “cell of origin” of different primary cancers. Finally, the ability to rapidly generate pathologically accurate liver and epithelial tumors with different genetic lesions makes the model an ideal preclinical system for testing new drugs or drug combinations for HCC and epithelial cancers (e.g., breast, lung, esophagus, ovarian, pancreatic, and head and neck cancers).

Using a cancer model of this invention, one can identify oncogenes that contribute to tumorigenesis. For example, using a HCC mouse model described herein, we identified the cellular inhibitor of apoptosis protein 1, cIAP1, as well as the yes-associated protein, Yap, as bona fide human oncogenes. Amplifications of mouse chromosome 9qA1 were observed at a high frequency in tumors derived from Myc-expressing cells, and at a lower frequency at the syntenic region of chromosome 11q22 in human liver cancers. Through comparative oncogenomics and expression analyses, we pinpointed cIAP1 and Yap as the driver genes of these regions in human liver cancer.

Although the 11q22 amplicon has been observed in several tumor types, previous studies have not agreed on the likely driver gene(s), in part, because validation was lacking. However, by returning to our liver cancer mouse model, we could directly test the oncogenic capabilities of cIAP1 and Yap in the genetic setting where spontaneous amplification occurred, and thus validate the lesions as a causative in HCC. By way of example, we showed that overexpression of each gene alone accelerated tumor growth when co-expressed with Myc, and conversely that knockdown of each gene in Myc-expressing tumors harboring the 9qA1 amplicon delayed tumor growth.

We have also shown that cIAP1, cIAP2 and Yap are required for rapid tumor growth. In other words, these genes are important for tumor maintenance, including progression. We have further showed that while cIAP1 and Yap can act independently as oncogenes, they synergize in transforming hepatoblasts and promoting tumorigenesis. We also showed that Yap and cIAP1 are more broadly active in just HCC. For example, Yap, alone or with cIAP1, can transform NIH 3T3 cells, which form sarcoma (sarcomagenesis) when transplanted subcutaneously into recipient mice. Based on the results of the present invention, we discovered that Yap and cIAP1 cooperate to drive sarcomagenesis more aggressively than either Yap or cIAP1 alone.

The 11q22 amplicon has been found in many cancers including lung, esophagus, ovarian, pancreatic, breast, and head and neck cancers. Thus, targeted therapies directed at cIAP1, cIAP2 and/or Yap are especially useful in treating these cancers and any other cancers that involve an 11q22 amplicon. Because cIAP1 and Yap are cooperating oncogenes, cancer therapies that concomitantly target these two proteins are especially effective. Molecules useful for cIAP/Yap targeted therapies include interfering RNA molecules (see below), antisense, small molecules, peptides, peptide mimetics, antibodies, etc. cIAP inhibitors are known in the art and include, without limitation, the small molecules, Smac/DIABLO mimetics and antisense oligonucleotides described in Wright & Duckett, supra, Schimmer, “Inhibitor of apoptosis proteins: translating basic knowledge into clinical practice,” Cancer Research 64, 7183-7190 (2004), and Schimmer et al., “Targeting the IAP family of caspase inhibitors as an emerging therapeutic strategy,” Hematology 2005, 215-219.

Using gene expression profiling on Yap-transformed NIH 3T3 cells and murine liver tumors harboring Yap amplifications, we also showed that overexpression of Yap upregulated many players in cell cycle regulation. For example, we found that Yap upregulated cyclin D and cyclin E (32 fold up in Yap-expressing NIH 3T3 cells), and that suppression of Yap using RNAi reduced cyclin E levels. Overexpression of Yap also upregulated many genes in the Ras-MAPK and MAPK signaling pathways. The Ras pathway is an essential signal transduction cascade that controls cell survival, growth, differentiation and transformation. Ras stimulates Raf activity, which then activates MAPK/ERK kinase (MEK). This in turn activates ERK. ERK regulates downstream signaling complexes of transcription factors that affect gene expression, rearrangements of the cytoskeleton and metabolism. ERK acts to coordinate responses to extracellular signals which results in the regulation of proliferation, differentiation, senescence, and apoptosis. One or more activating genetic mutations of the components of this pathway have been found to be associated with cancers (Thompson and Lyons, “Recent progress in targeting the Raf/MEK/ERK pathway with inhibitors in cancer drug discovery,” Current Opinion in Pharmacology 5:350-356 (2005)). Thus, Yap modulates the expression of cell cycle genes and genes in the MAPK pathway.

Thus, a therapeutic paradigm that targets cIAP1, cIAP2, and/or Yap as well as the proteins that are upregulated by them in cell cycle control and/or the Ras-MAPK signaling pathway are useful in treating cancers involving an 11q22 amplicon (or otherwise amplification of the cIAP1, cIAP2 and Yap genes). To treat a cancer patient who displays genomic amplification in the 11q22 region, inhibitors of cIAP1, cIAP2 and/or Yap can be used in conjunction with an inhibitor of MEK or CDK. Inhibitors of MEK and CDK are known in the art. Inhibitors of MEK include, without limitation, Sorafenib, PD0325901, and AZD6244, all of which are described in Thompson and Lyons, supra, and derivatives thereof. Inhibitors of CDK include, without limitation, Flavopiridol, 7-hydroxystaurosporine, Bryostatin-1, R-roscovitine, N-acyl-2-aminothiazole analogs, and imidazopyridines, all of which are described in Schwartz and Shah, “Targeting the cell cycle: a new approach to cancer therapy,” Journal of Clinical Oncology 23:9408-9421 (2006). In some embodiments, an IAP or Yap inhibitor can be used in conjunction with a cancer therapy targeting growth factors and their receptors such as EGFR (e.g., a Gefitinib, Erlotinib, or Imatinib therapy).

In further embodiments, an IAP or Yap inhibitor can be used in conjunction with chemotherapy agents specifically selected for a particular cancer. Such agents include, without limitation, folate antagonists, pyrimidines, purine antimetabolites, alkylating agents, platinum antitumor compounds, DNA interchelators, and microtubule targeting compounds. In still further embodiments, an IAP or Yap inhibitor can be used in conjunction with anti-angiogenic agents and anti-metastatic agents. In any of the above combination therapies, the various drugs can be administered concurrently or sequentially.

We have made the important discovery that two genes embedded within the same focal amplification can cooperate in cancer development. This discovery forces a re-evaluation of other more well established amplified loci in human genomes and identify additional therapeutic targets. For example, one can examine regions containing established oncogene targets (e.g., erbB2, cyclin D, etc.) and identify additional therapeutic targets.

As used herein, a non-human animal includes any animal, other than a human. Examples of such non-human animals include without limitation: aquatic animals, e.g., fish, sharks, dolphins and the like; farm animals, e.g., pigs, goats, cows, horses, rabbits and the like; rodents, e.g., rats, guinea pigs and mice; non-human primates, e.g., baboons, chimpanzees and monkeys; and domestic animals, e.g., cats and dogs. Rodents are preferred. Mice are more preferred.

The non-human animals can be wild type or can carry genetic alterations. For example, they may be immunocompromised or immunodeficient, e.g., a severe combined immunodeficiency (SCID) animal.

As used herein, hepatocytes include all descendants of embryonic liver progenitor cells. Preferably, primary hepatocytes are used in the methods and models of this invention. Primary hepatocytes from adult non-human animals or embryonic liver progenitor cells can be isolated using standard and conventional protocols. In short term primary culture the hepatocytes can be virally transduced with vectors carrying oncogenes and/or expression cassettes for short hairpin RNAs directed against tumor suppressor genes. Such transductions may be effected using standard and conventional protocols.

The term vector refers to a nucleic acid molecule capable of transporting another nucleic acid to which it has been linked. One type of vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in a host cell into which they are introduced (e.g., vectors having an origin of replication which functions in the host cell). Other vectors can be integrated stably into the genome of a host cell upon introduction into the host cell, and are thereby replicated along with the host genome. Preferred viral vectors include retroviral and lentiviral vectors. Moreover, certain preferred vectors are capable of directing the expression of nucleic acid sequences to which they are operatively linked. Such vectors are referred to herein as recombinant expression vectors or simply, expression vectors. Preferably, the vector carries marker cassettes, more preferably, GFP expression cassettes, so that the course of transduction, engrafting and tumor growth and remission may be observed. Preferably, the vector also carries a ubiquitous promoter to permit expression or up-regulation of oncogenes in all cell types of epithelium (i.e., stem cell and non-stem cell compartments).

As used herein, viral transduction refers to a general method of gene transfer. As embodied herein, viral transduction is used for establishing stable expression of genes in culture. Viral transduction and long-term expression of genes in cells, preferably cultured hepatocytes, is preferably accomplished using viral vectors.

After viral transduction the cells are preferably injected into the spleen of the recipient non-human animal, preferably a rodent and most preferably a mouse, that are preferably pretreated with a liver cell cycle inhibitor. Using this approach, the genetically modified or altered hepatocytes migrate via the portal vein into the recipient liver and engraft the organ. An additional proliferation stimulus to the liver can preferably be given after hepatocyte transplantation by serial administration of CCl₄.

Non-human animals harboring hepatocellular carcinomas of different genetic constellations produced by the altered hepatocytes can be characterized with regard to time to tumor onset and survival time. Tumors of different genetic constellations can also be histologically examined and classified by experienced pathologists.

As used herein, an altered hepatocyte refers to a change in the lever of a gene and/or gene product with respect to any one of its measurable activities in a hepatocyte (e.g., the function which it performs and the way in which it does so, including chemical or structural differences and/or differences in binding or association with other factors). An altered hepatocyte may be effected by one or more structural changes to the nucleic acid or polypeptide sequence, a chemical modification, an altered association with itself or another cellular component or an altered subcellular localization. Preferably, an altered hepatocyte may have “activated” or “increased” expression of an oncogene, “repressed” or “decreased” expression of a tumor suppressor gene or both.

The increased expression of an oncogene refers to a produced level of transcription and/or translation of a nucleic acid or protein product encoded by an oncogenic sequence in a cell. Increased expression or up regulation of an oncogene can be non-regulated (i.e., a constitutive “on” signal) or regulated (i.e., the “on” signal is induced or repressed by another signal or molecule within the cell). An activated oncogene can result from, e.g., over expression of an encoding nucleic acid, an altered structure (e.g., primary amino acid changes or post-transcriptional modifications such as phosphorylation) which causes higher levels of activity, a modification which causes higher levels of activity through association with other molecules in the cell (e.g., attachment of a targeting domain) and the like.

The decreased expression of a tumor suppressor gene refers to an inhibited, inactivated or down regulated level of transcription and/or translation of a nucleic acid or protein product encoded by a tumor suppressor gene sequence in a cell. Reduced expression of a tumor suppressor gene can be non-regulated (i.e., a constitutive “off” signal) or regulated (i.e., the “off” signal is activated or repressed by another signal or molecule within the cell). As preferred herein, a repressed tumor suppressor gene can result from inhibited expression of an encoding nucleic acid (e.g., most preferably a short hairpin RNA using RNA interference approaches, see supra). Reduced expression of a tumor suppressor gene can also result from an altered structure (e.g., primary amino acid changes or post-transcriptional modifications such as phosphorylation) which causes reduced levels of activity, a modification which causes reduced levels of activity through association with other molecules in the cell (e.g., binding proteins which inhibit activity or sequestration) and the like.

A short hairpin RNA refers to a segment of RNA that is complementary with a portion of one or more target genes (i.e. complementary with one or more transcripts of one or more target genes). When a nucleic acid construct encoding a short hairpin RNA is introduced into a cell, the cell incurs partial or complete loss of expression of the target gene. In this way, a short hairpin RNA functions as a sequence specific expression inhibitor or modulator in transfected cells. The use of short hairpin RNAs facilitates the down-regulation of tumor suppressor genes and allows for analysis of hypomorphic alleles. The short hairpin RNAs that are useful in the invention can be produced using a wide variety of RNA interference (“RNAi”) techniques that are well known in the art. The invention may be practiced using short hairpin RNAs that are synthetically produced as well as microRNA (miRNA) molecules that are found in nature and can be remodeled to function as synthetic silencing short hairpin RNAs. A preferred embodiment of the invention is the use of a short hairpin RNA that mediates inhibition of a oncogenic signal, preferably a tumor suppressor gene and thus apoptotic signaling in a cell. Preferably, the short hairpin RNAs are against cIAP1, cIAP2, or Yap.

Exemplary shRNAs against cIAP1 are:

1. Hairpin sequence:
(SEQ ID NO: 1)
TGCTGTTGACAGTGAGCGAGCCAAGGTACCTTACACCAAATAGTGAAGC
CACAGATGTATTTGGTGTAAGGTACCTTGGCCTGCCTACTGCCTCGGA
Mature product:
(SEQ ID NO: 2)
CCAAGGTACCTTACACCAA
2. Hairpin sequence:
(SEQ ID NO: 3)
TGCTGTTGACAGTGAGCGCGGACTTCTTGGATTTGGAATTTAGTGAAGC
CACAGATGTAAATTCCAAATCCAAGAAGTCCTTGCCTACTGCCTCGGA
Mature product:
(SEQ ID NO: 4)
GACTTCTTGGATTTGGAAT
3. Hairpin sequence:
(SEQ ID NO: 5)
TGCTGTTGACAGTGAGCGCGCTCTAGCCCTCTTAATTCTATAGTGAAGC
CACAGATGTATAGAATTAAGAGGGCTAGAGCATGCCTACTGCCTCGGA
Mature product:
(SEQ ID NO: 6)
CTCTAGCCCTCTTAATTCT
4. Hairpin sequence:
(SEQ ID NO: 7)
TGCTGTTGACAGTGAGCGCGGAAATTGACTCCACGTTATATAGTGAAGC
CACAGATGTATATAACGTGGAGTCAATTTCCTTGCCTACTGCCTCGGA
Mature product:
(SEQ ID NO: 8)
GAAATTGACTCCACGTTAT

Exemplary shRNAs against cIAP2 are:

1. Hairpin sequence:
(SEQ ID NO: 9)
TGCTGTTGACAGTGAGCGACCGTATTAGAACATTCTCTAATAGTGAAGC
CACAGATGTATTAGAGAATGTTCTAATACGGGTGCCTACTGCCTCGGA
Mature product:
(SEQ ID NO: 10)
CGTATTAGAACATTCTCTA
2. Hairpin sequence:
(SEQ ID NO: 11)
TGCTGTTGACAGTGAGCGACCTGCGTTATACAGAGATATATAGTGAAGC
CACAGATGTATATATCTCTGTATAACGCAGGGTGCCTACTGCCTCGGA
Mature product:
(SEQ ID NO: 12)
CTGCGTTATACAGAGATAT
3. Hair sequence:
(SEQ ID NO: 13)
TGCTGTTGACAGTGAGCGCGCACTAATCCGGAAGAACAAATAGTGAAGC
CACAGATGTATTTGTTCTTCCGGATTAGTGCTTGCCTACTGCCTCGGA
Mature product:
(SEQ ID NO: 14)
CACTAATCCGGAAGAACAA
4. Hair sequence:
(SEQ ID NO: 15)
TGCTGTTGACAGTGAGCGCGCTTTGCAAGTTCTGAGAATATAGTGAAGC
CACAGATGTATATTCTCAGAACTTGCAAAGCTTGCCTACTGCCTCGGA
Mature product:
(SEQ ID NO: 16)
CTTTGCAAGTTCTGAGAAT

Exemplary shRNAs against Yap are:

1. Hairpin sequence:
(SEQ ID NO: 17)
TGCTGTTGACAGTGAGCGCGCACCCTGACTCTCACTAAATTAGTGAAGC
CACAGATGTAATTTAGTGAGAGTCAGGGTGCTTGCCTACTGCCTCGGA
Mature product:
(SEQ ID NO: 18)
CACCCTGACTCTCACTAAA
2. Hairpin sequence:
(SEQ ID NO: 19)
TGCTGTTGACAGTGAGCGCGGAGATGCAATGAACATAGAATAGTGAAGC
CACAGATGTATTCTATGTTCATTGCATCTCCTTGCCTACTGCCTCGGA
Mature product:
(SEQ ID NO: 20)
GAGATGCAATGAACATAGA
3. Hairpin sequence:
(SEQ ID NO: 21)
TGCTGTTGACAGTGAGCGAGCAGACAGATTCCTTTGITAATAGTGAAGC
CACAGATGTATTAACAAAGGAATCTGTCTGCGTGCCTACTGCCTCGGA
Mature product:
(SEQ ID NO: 22)
CAGACAGATTCCTTTGTTA

Other methods of RNA interference may also be used in the practice of this invention. See, e.g., Scherer and Rossi, Nature Biotechnology 21:1457-65 (2003) for a review on sequence-specific mRNA knockdown of using antisense oligonucleotides, ribozymes, DNAzymes, RNAi and siRNAs. See also, International Patent Application PCT/US2003/030901 (Publication No. WO 2004/029219 A2), filed Sep. 29, 2003 and entitled “Cell-based RNA Interference and Related Methods and Compositions.” These RNA molecules can be introduced into the patient using an expression vector at a cancer site or systemically.

As used herein the term liver or hepatocellular cancer tumor refers to a group of cells which are committed to a hepatocellular lineage and which exhibit an altered growth phenotype. The term encompasses tumors that are associated with hepatocellular malignancy (i.e., HCC) as well as with pre-malignant conditions such as hepatoproliferative and hepatocellular hyperplasia and hepatocellular adenoma, which include proliferative lesions that are perceived to be secondary responses to degenerative changes in the liver.

The non-human animals of the invention are useful in the study of the impact of genotype on pathology or treatment response in vivo. Thus, the methods and models of the invention have implications for understanding disease progression in human liver carcinomas of specific genetic origin. The invention is also useful for determining the efficacy of a therapy in treating liver cancer. For example, a potential therapy may be administered to a non-human animal, produced by the methods embodied herein, and the non-human animal monitored for liver tumor formation, growth, progression or remission. Often, increased time to tumor formation or growth indicates sensitivity of the tumor to the therapy.

Genomic analysis of human carcinomas can be performed by gene expression profiling, e.g., ROMA. Such analysis in the tumors produced according to the invention has revealed a low signal to noise ratio of profiled genes, suggesting that the majority of detected genetic alterations in human tumors (having a high signal to noise ratio) may not be originally involved in tumor development but may be a by-product of tumor development. The analysis of mouse tumors produced according to the invention has shown that these tumors have a low signal to noise ratio, suggesting that a higher proportion of the identified lesions are specifically involved in tumor initiation/progression. Thus, the analysis of mouse tumors by gene expression profiling can serve as a filter for the “noisy” human tumors. Results obtained from mouse profiling using ROMA can be aligned with ROMA data obtained from human hepatocellular carcinomas. Overlapping amplifications or deletions then can be prioritized for further evaluation.

Tumors showing specific amplifications of candidate oncogenes in gene expression profiles can be outgrown in culture. Using stable RNAi, efficient knockdown of these genes can be achieved. Tumor cells with stable knockdown of a previously amplified gene can be re-transplanted into the mouse model of the current invention. Using this approach new therapeutic targets for hepatocellular carcinoma and related carcinomas can be obtained and the specific consequences of knocking down an amplified gene with regard to tumor growth or metastases can be studied. Drug therapies that specifically inhibit the identified targets can be developed.

Therapies that may be tested and evaluated in the methods and models of this invention include both general and targeted therapies. As used herein, a general therapy can be, for example, a pharmaceutical or chemical with physiological effects, such as pharmaceuticals that have been used in chemotherapy for cancer. Chemotherapeutic agents inhibit proliferation of tumor cells, and generally interfere with DNA replication or cellular metabolism. See, e.g., The McGraw-Hill Dictionary of Chemical Terms (Parker, S., Ed., McGraw-Hill, San Francisco (1985)). Chemotherapeutic agents may or may not have been characterized for their target of action in cells. However, this invention and its methods and models allow evaluation of such therapies for defined genetic alterations.

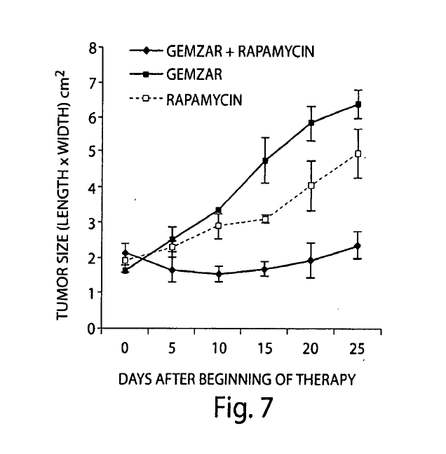

A targeted therapy refers to a therapy that directly interferes with a specific gene Preferably, a targeted therapy directly interferes with the expression of a gene involved in liver cancer. The effectiveness of a targeted therapy can be determined by the ability of the therapy to inhibit an oncogene or activate a tumor suppressor gene. Most preferably, the therapies are used and evaluated in combination. For example, as shown in FIG. 7, upon onset of liver cancer tumors, animals can be treated with Gemcitabine (“Gemzar”), a chemotherapeutic agent that is an anti-metabolite that functions as a mild chemotherapeutic to interfere with the growth of cancer cells. As shown in FIG. 7, it has virtually no effect on tumor growth of the particular tumor tested in FIG. 7. The tumor can also be treated with Rapamycin, a targeted therapy that inhibits the mammalian target of rapamycin (mTOR), which has some effect on the tumor growth. In combination, however, as depicted in FIG. 7, the two therapies control tumor growth.

The size and growth of tumors after therapy can be monitored by a wide variety of ways known in the art. Preferably, whole body fluorescence imaging is used because the preferred viral vectors of this invention carry a GFP expression cassette. See, e.g., Schmitt et al., “Dissecting p53 tumor suppressor functions in vivo,” Cancer Cell 1:289-98 (2002). Tumors can also be examined histologically. Paraffin embedded tumor sections can be used to perform immunohistochemistry for cytokeratins and ki-67 as well as TUNEL-staining. The apoptotic rate of hepatocytes can be analyzed by TUNEL assay according to published protocols. Di Cristofano et al., “Pten and p27KIP1 cooperate in prostate cancer tumor suppression in the mouse,” Nature Genetics, 27:222-224 (2001).

Beyond having important implications for understanding liver cancer, the evaluations and observations made possible by the methods and models of this invention provide insight into the utility of targeted approaches in cancer therapy.

Throughout this specification and embodiments, the word “comprise” or variations such as “comprises” or “comprising” will be understood to imply the inclusion of a stated integer or group of integers but not the exclusion of any other integer or group of integers.

The following examples are meant to illustrate the methods and materials of the present invention. Suitable modifications and adaptations of the described conditions and parameters normally encountered in the art are within the spirit and scope of the present invention.

### Example 1

### Generation and Transplantation of Genetically Altered Liver Progenitor Cells

This example describes the generation and transplantation of recombinant embryonic hepatoblasts. Embryonic hepatoblasts express high E-Cadherin levels on their cell surface and thus can be isolated to high purity from fetal livers using magnetic bead selection (Nitou et al., “Purification of fetal mouse hepatoblasts by magnetic beads coated with monoclonal anti-e-cadherin antibodies and their in vitro culture,” Exp. Cell Res. 279, 330-343 (2002)). These cells express markers characteristic of bi-potential oval cells, the presumed cellular target of transformation in the adult rodent liver (Thorgeirsson, “Hepatic stem cells in liver regeneration,” FASEB J. 10, 1249-1256 (1996); Alison and Lovell, “Liver cancer: the role of stem cells,” Cell Prolif. 38, 407-421 (2005)).

Although these cells proliferated poorly in initial experiments, the introduction of defined medium (Block et al., “Population expansion, clonal growth, and specific differentiation patterns in primary cultures of hepatocytes induced by 1-IGF/SF, EGF and TGF alpha in a chemically defined (HGM) medium,” J. Cell Biol. 132, 1133-1149 (1996)), feeder layers, and gelatin coated plates to the culture conditions enabled the hepatoblasts to be expanded without loss of their defining characteristics.

These conditions also allowed efficient gene transfer using MSCV-based retroviral vectors expressing green fluorescent protein (GFP) reporter gene or short-hairpin RNAs (shRNAs) capable of suppressing gene expression through RNA interference.

To determine whether genetically modified hepatoblasts could colonize recipient livers, a protocol was used that optimized engraftment of transplanted cells in the recipient liver (Guo et al., “Liver repopulation after cell transplantation in mice treated with retrorsine and carbon tetrachloride,” Transplantation 73, 1818-1824 (2002)). Animals were pretreated with retrorsine, an alkaloid that exerts a strong and persistent block of native hepatocyte proliferation and increases the competitive advantage of transplanted cells. Ten days after the last retrorsine treatment, 2×10⁶ GFP-tagged E-Cadherin⁺ liver progenitor cells were delivered to the liver by intrasplenic injection.

One week after injection, immunohistochemical analysis of liver sections revealed that approximately one percent of the host liver consisted of “seeded” GFP-positive cells that were embedded within the normal liver architecture. Transplanted hepatoblasts engrafted the recipient livers and were morphologically indistinguishable from the host hepatocytes (H&E). Immunofluorescence with a primary antibody directed against GFP allowed for detection of the transplanted hepatocytes. Intrahepatic liver carcinomas were detected by whole body, external GFP-tumor imaging or by direct imaging of the respective explanted tumor bearing livers. The resulting tumors were visualized by either external GFP imaging or direct GFP-imaging of the explanted liver.

### Example 2

Generation of Liver Carcinomas from Transplanted Liver Progenitor Cells

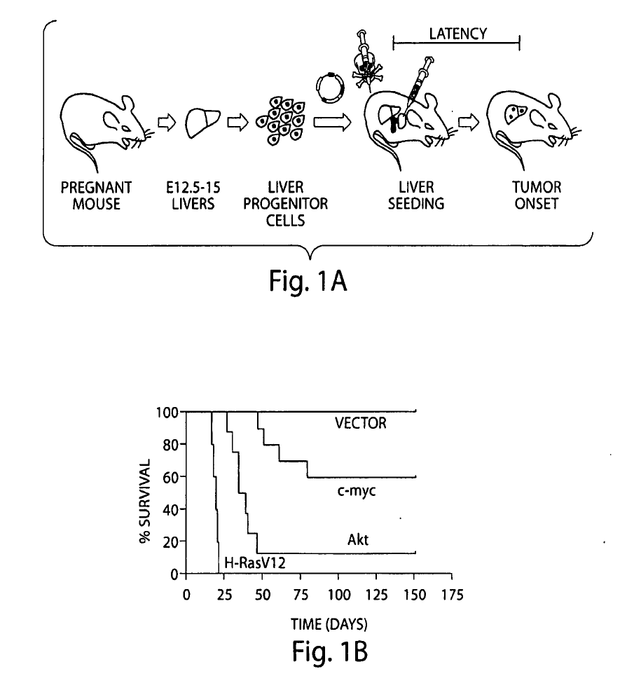

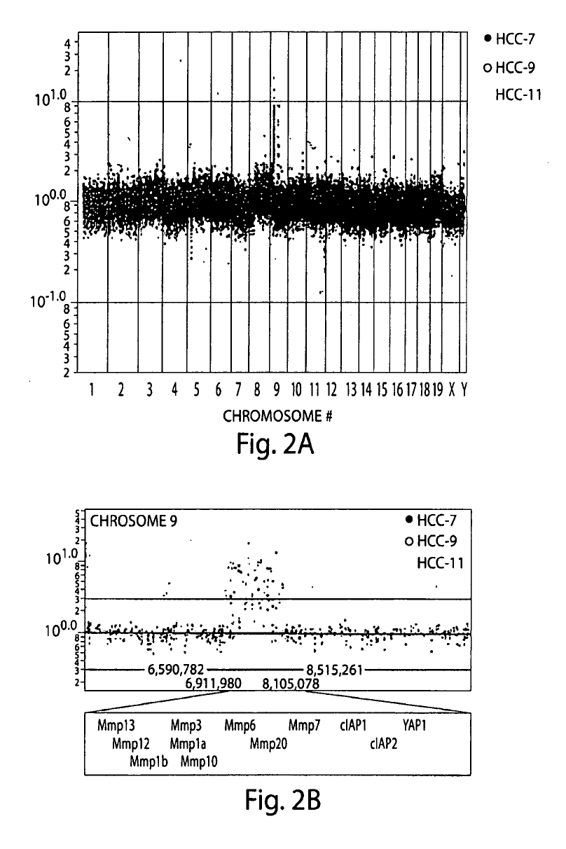

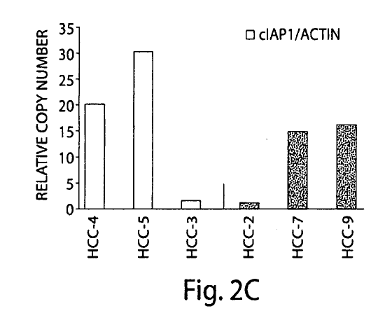

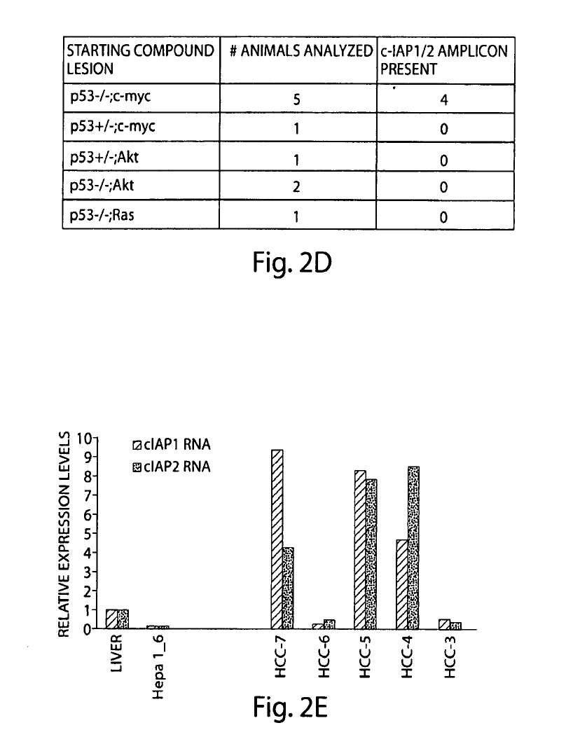

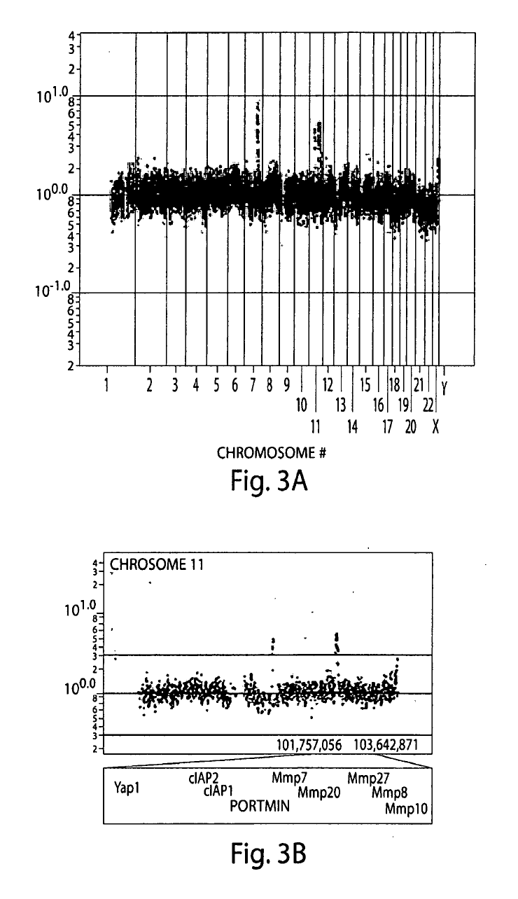

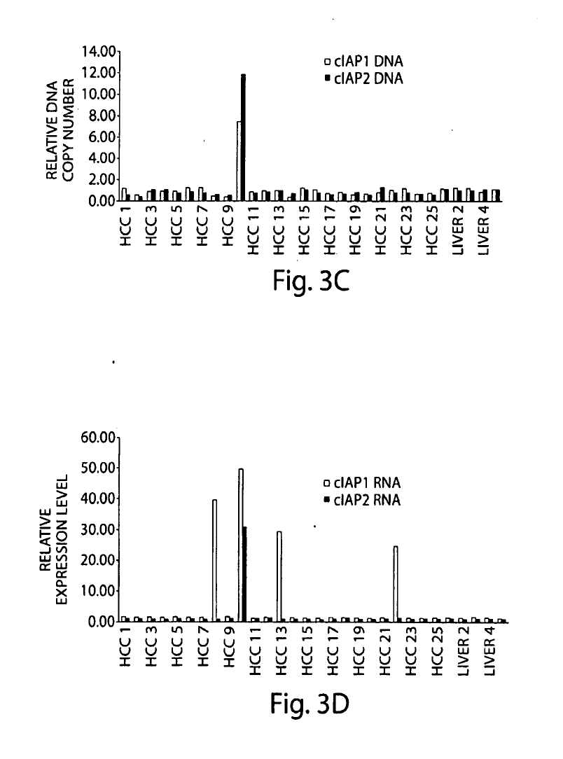

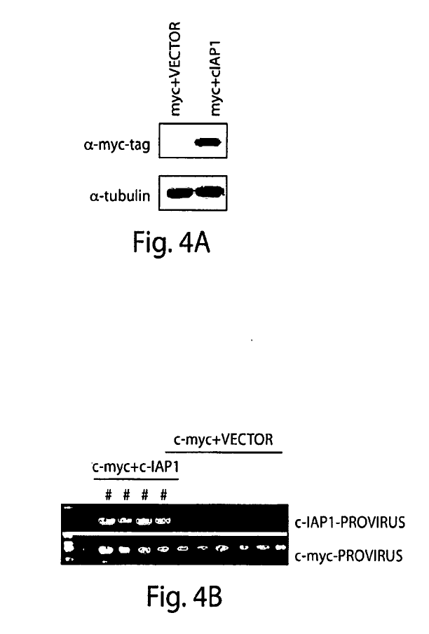

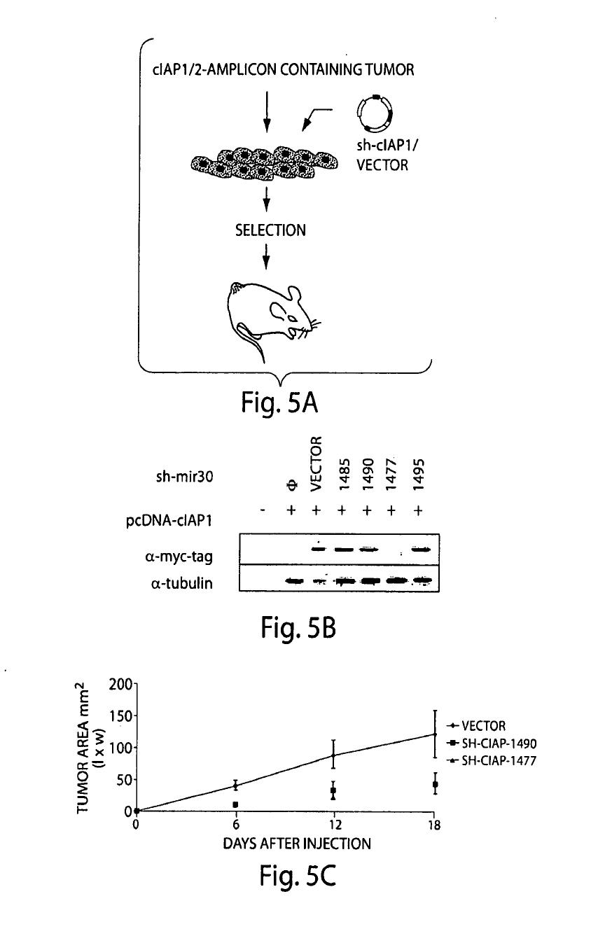

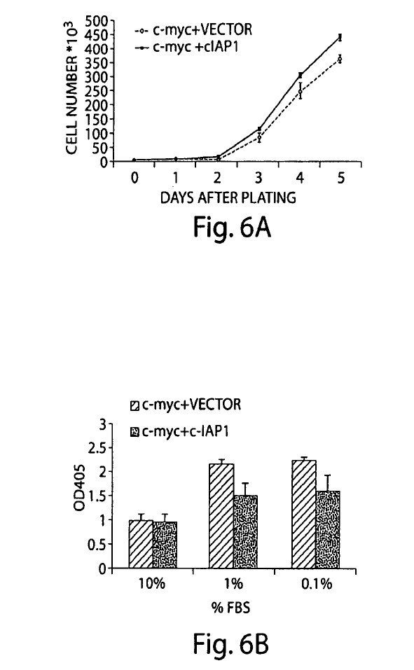

Hepatoblasts were isolated from p53−/−fetal livers and the cells were transduced with retroviruses co-expressing different oncogenes: Myc (c-myc), activated Akt (Akt1), or oncogenic Ras (H-rasV12) (each of which affect signaling pathways altered in human liver cancer) and a GFP reporter, to give rise to orthotopic liver carcinomas after intrahepatic seeding. As above, these p53 deficient liver progenitor transduced cell populations were transplanted into retrorsine treated mice (see FIG. 1A). To further facilitate expansion of the transplanted cells, recipient mice were treated with CCl₄ (Guo et al., supra) and monitored for signs of disease by abdominal palpation of the liver and whole body fluorescence imaging. Although p53−/−hepatoblasts were not tumorigenic during the time frame of analysis, each of the cell populations that also expressed an oncogene eventually produced GFP-positive tumors in the livers of recipient mice. The intrahepatic liver carcinomas were detected by whole body, external GFP-tumor imaging.

Gross pathological analysis of explanted livers revealed that Myc-expressing tumors differed significantly from those expressing Akt or Ras. First, Myc-expressing tumors grew primarily as unilocular tumors, whereas Akt- and Ras-derived tumors showed aggressive, multilocular and infiltrative intrahepatic growth. Second, the innate tumorigenicity of p53−/−liver progenitor cells expressing Myc was significantly lower than those expressing Akt or Ras; thus, Akt or Ras triggered the development of liver carcinomas with an efficiency of nearly 100%, while Myc produced tumors at a penetrance around 40% (FIG. 1B). Of note, p53 loss clearly contributed to tumorigenesis, since tumors arising in mice reconstituted with p53+/−hepatoblasts showed further delayed tumor onset and loss of the wild-type p53 allele. In most instances, GFP-positive cells derived from these tumors could be readily grown in culture, and subsequently formed secondary tumors upon subcutaneous injection into immunocompromised mice or direct intrahepatic injection into syngeneic recipients.

### Example 3

### Murine Liver Carcinomas Histopathologically Resemble Features of Human HCC

To determine whether the murine tumors produced from liver progenitors resemble human liver cancer, a panel of hematoxylin/eosin (H&E) stained sections derived from primary Myc-induced murine hepatomas were examined by an experienced liver pathologist. These tumors were classified as moderately well to poorly differentiated HCCs with a mostly solid, sometimes mixed solid/trabecular growth pattern. A smaller proportion of tumors revealed growth patterns resembling trabecular or pseudoglandular HCC. All tumors examined stained positive for cytokeratin 8, confirming their liver origin. However, despite their derivation from cytokeratin 19 positive liver progenitor cells, most HCCs lost this marker during tumorigenesis. The tumors also expressed high albumin levels and similar to the situation in human HCC. About half were positive for alpha-fetoprotein; most also expressed moderate levels of vimentin, a marker linked to aggressive tumor behavior (Hu et al., “Association of Vimentin overexpression and hepatocellular carcinoma metastasis,” Oncogene 23, 298-302 (2004)). Furthermore, transplanted tumors retained their HCC histology when injected orthotopically into the liver, or subcutaneously into immunocompromised mice. These findings confirm that ex vivo manipulated liver progenitor cells produce tumors that recapitulate the histopathology of human HCC.

### Example 4

### ROMA Identifies Spontaneous Mutations in a Subset of Murine Liver Carcinomas

Epithelial cancers require a series of genetic alterations during clonal evolution to an advanced disease. To molecularly characterize the murine HCCs described above, spontaneously acquired lesions in those cancers were analyzed using representational oligonucleotide microarray analysis (ROMA), a genome-wide scanning method capable of identifying copy number alterations in tumor cells at high resolution (Lucito et al., “Representational oligonucleotide microarray analysis: a high-resolution method to detect genome copy number variation,” Genome Res. 13, 2291-2305 (2003); Sebat et al., “Large-scale copy number polymorphism in the human genome,” Science 305, 525-528 (2004)). Each human or mouse ROMA array consisted of 85,000 oligonucleotide probes designed to the UCSC Apr/2003 draft assembly of the human genome and the UCSC Feb/2003 draft assembly of the mouse genome, allowing genome scanning at a theoretical resolution of approximately 35 kb.

Genomic representations were produced from DNA obtained from several murine liver tumors and from normal mouse tissue of the same genetic background. The representations were fluorescently labeled and hybridized to the ROMA microarrays. The data derived after scanning were normalized as described (Sebat et al., supra). ROMA identified localized DNA amplifications in murine HCCs. Although we did not detect focal genomic alterations (5 Mb) in liver cancers induced by Akt, a number were detected in those initiated by Myc or Ras. For example, a ras-expressing tumor harbored two focal amplifications on chromosome 15, including a 250 Kb amplicon containing Rnf19 and a 2 Mb amplicon containing c-myc. Single probe resolution of chromosome 15 revealed increased copy number for Rnf19 and myc. While Rnf19 had not been previously linked to tumorigenesis, c-myc amplification is a common event in human liver cancer (Peng et al., “Amplification of the c-myc gene in human hepatocellular carcinoma: biologic significance,” J. Formos. Med. Assoc. 92, 866-870 (1993)). Furthermore, c-myc cooperates with oncogenic ras in transgenic models of HCC (Sandgren et al., “Oncogene-induced liver neoplasia in transgenic mice,” Oncogene 4, 715-724 (1989)). That a mutation affecting an established liver oncogene can occur spontaneously in these tumors underscores the relevance of the model of the present invention, and indicates that further analyses would reveal other genes involved in human cancer.

ROMA was performed on seven independent p53−/−; myc, six independent p53−/−; Ras and six independent p53−/−; Akt derived liver tumors. Summarized are the gene copy number alterations found in the tumors that had changes. No focal genomic alterations were found in p53−/−; Aid derived liver tumors. Also presented are the representative genes contained in focal genomic amplicons found by ROMA in murine tumors derived from p53−/−; myc hepatoblasts, except for those denoted with an * which were detected in p53−/−; Ras derived tumors. For the recurrent amplicons “size” is the minimal overlap between the individual amplicons. Four myc driven HCCs had overlapping 9qA1 amplicons and the genomic coordinates of those are listed below the table. Data from the ROMA analysis are shown in the following table.

SUPPLEMENTARY TABLE 1
Chromosomal positions and frequency of genomic amplifications found
in murine hepatomas as determined by ROMA. All murine tumors were
derived from p53−/−; myc hepatoblasts except for those denoted
by an * which were derived from p53−/−; ras hepatoblasts

| Chr. | Start | Size (Mb) | Freq. | Total Genes | Representative Genes |
|---|---|---|---|---|---|
| 4 | 80.2 | 2.5 | 1 | 6 | Nfib |
| 7 | 117.6 | 0.6 | 1 | 3 | Fgfr2 |
| 7 | 126.5 | 0.6 | 1 | 4 | Stk32c |
| 8 | 10.9 | 0.8 | 1 | 5 | Rab20 |
| 8 | 14.1 | 1.5 | 1 | 3 | Arhgef10 |
| 8 | 32.6 | 1.7 | 1 | 8 | Dusp4 |
| 8 | 39.0 | 1.5 | 1 | 14 | Pdgfrl |
| 8 | 83.0 | 1.1 | 1 | 13 | Rln3 |
| 9 | 3.3 | 0.9 | 1 | 1 | Cwf19l1 |
| 9 | 4.3 | 0.2 | 1 | 1 | Gria4 |
| 9 | 7.0 | 1.0 | 4 | 12 | Birc2; Birc3: Yap1 |
| 9 | 36.3 | 0.2 | 1 | 2 | Scn3b |
| *15 | 36.3 | 0.2 | 1 | 1 | Rnf19 |
| *15 | 61.0 | 2.1 | 1 | 2 | Myc |

Genomic coordinates of the 4 p53−/−: myc induced 9qA1 amplicons
chr9: 6,911,980-8,105,078
chr9: 6,590,782-8,515,261
chr9: 6,923,942-8,108,107
chr9: 6,565,908-8,760,915

### Example 5

### Recurrent Amplification of Chromosome 9qA1 in Myc-Expressing HCCs

ROMA analysis of seven independently derived Myc-expressing HCCs identified a focal amplicon on mouse chromosome 9qA1 in four of these tumors. Genome-wide profiles of three independent HCCs (Tu-7, Tu-9, Tu-13) derived from p53−/−; myc embryonic hepatoblasts were overlaid and confirmed the recurrent overlapping DNA amplification on chromosome 9. Single probe resolution of the amplicon on chromosome 9qA1 revealed that the minimal overlapping region was approximately 1 Mb and contained genes encoding for several matrix metalloproteinases (MMPs), Yap1, cIAP1 (Birc2), and cIAP2 (Birc3) as annotated in the UCSC genome browser. An EST to Porimin also mapped to this region. Amplification of this region was confirmed by genomic Q-PCR using a probe targeting the middle of the 9qA1 amplicon within the cIAP1 gene. Surprisingly, 9qA1 was never found amplified in Ras or Akt driven liver carcinomas as assessed by either genomic Q-PCR analysis or ROMA. These observations indicate that at least one of the genes in the 9qA1 region cooperates with myc and p53 loss to promote hepatocarcinogenesis.

### Example 6

Comparative Oncogenomics Reveals Lesions in Common between Murine and Human Cancers

In parallel to the analysis of murine HCCs, ROMA analysis was conducted on human HCC samples. These human tumors showed more complex alterations than their murine counterparts. Yet using a strict cut-off of <5 Mb, we were able to detect copy number alterations affecting genes previously linked to HCC. For example, three tumors had a chromosome 11 amplification containing CCND1 (cyclin D1), two had a chromosome 7 amplification containing c-MET, and one had a deletion of chromosome 9 harboring the CDKN2A (INK4a/ARF) locus. Interestingly, we also detected focal amplification of chromosome 11q22, a region that is syntenic to the murine 9qA1 locus.

ROMA identified amplification of the human syntenic region 11q22 in HCC and other cancers. A genome-wide profile of a human HCC revealed an amplification on chromosome 7 containing the c-MET gene and 3 regions amplified on chromosome 11. The sharply delineated regions of amplification on chromosome 11 included CCND1, B′ (containing no known genes), and 11q22 (containing, inter alia, Yap1, Porimin, cIAP2, cIAP1 and several matrix metalloproteinases (MMPs)). We found a second 11q22 amplicon in a subsequently analyzed set of 23 additional human HCCs. ROMA results were verified by genomic Q-PCR analysis using probes to the cIAP1 and cIAP2 loci. We also detected this same amplicon four times in a set of 53 human esophageal cancers, indicating that it occurs in gastrointestinal malignancies derived from developmentally related organs. A single probe resolution of chromosome 11 of a representative esophageal tumor revealed that 11q22 contains same genes as identified above. Likewise, a genome-wide profile of an ovarian carcinoma also revealed chromosome 11 amplification. Much like the chromosome 9 amplicon in murine HCCs, the boundaries of the 11422 amplicon in human HCCs and esophageal cancers included genes encoding several matrix metalloproteinases (MMPs), Porimin, Yap1, cIAP1 and cIAP2. A single probe resolution of the 11q22 amplicon showed a lack of amplification of the MMP cluster but amplification in the region including CnFn5, Pgr, Trpc6, Porimin, Yap1, cIAP1 and cIAP2.

The human 11q22 amplicon has previously been observed at low frequency in other human cancers, although no driver gene has been decisively identified (Imoto et al., “Identification of cIAP1 as a candidate target gene within an amplicon at 11q22 in esophageal squamous cell carcinomas,” Cancer Res. 61, 6629-6634 (2001); Dai et al., “A comprehensive search for DNA amplification in lung cancer identifies inhibitors of apoptosis cIAP1 and cIAP2 as candidate oncogenes,” Hum. Mol. Genet. 12, 791-801 (2003); Bashyam et al., “Array-based comparative genomic hybridization identifies localized DNA amplifications and homozygous deletions in pancreatic cancer,” Neoplasia. 7, 556-562 (2005); and Snijders et al., “Rare amplicons implicate frequent deregulation of cell fate specification pathways in oral squamous cell carcinoma,” Oncogene 24, 4232-4242 (2005)). While it represents only one of many low frequency events in these tumors, our cross-species comparison indicates that certain genes within this recurrent amplified region are crucial for tumorigenesis.

Chromosomal position and frequency of genomic alterations in 26 human HCCs was determined by ROMA. Data from the ROMA analysis of human HCC specimen are shown in the following table. (A) Amplified regions, frequency and representative genes found in human HCC samples. For recurrent amplicons, “size” depicts the minimal overlap of the individual amplicons. Individual breakpoints for recurrent amplicons were (start-end): c-MET amplicon 1=113412074-116125069, c-MET amplicon 2=114695639-116125069, CCND1 amplicon 1=67588736-70378610, CCND1 amplicon 2=68917231-69274977, CCND1 amplicon 3=68970345-69301635, BIRC2(cIAP1)/Yap amplicon 1=100986325-102427746, BIRC2(cIAP1)/Yap amplicon 2=101257690-103086048. (B) Deleted regions, frequency and representative genes found in human HCC samples. For recurrent deletions “size” depicts the minimal overlapping region. Individual breakpoints for recurrent deletions were: PTEN deletion 1=89690492- 91553410, PTEN deletion 2=89288663-90909401.

SUPPLEMENTARY TABLE 2
Chromosomal positions and frequency of genomic amplifications (A)
and deletions (B) found in 26 human HCCs as determined by ROMA.

| | | | | | |
|---|---|---|---|---|---|
| Chr. | Start | Size (Mb) | Freq. | Total Genes | Representative Genes |

A

| | | | | | |
|---|---|---|---|---|---|
| 1 | 22.3 | 0.9 | 1 | 7 | EPHB2; EPHA8 |
| 1 | 166.5 | 4.2 | 1 | 31 | |
| 1 | 241.7 | 3.7 | 1 | 61 | |
| 2 | 211.1 | 0.5 | 1 | 2 | LANCL1 |
| 3 | 20.8 | 2.6 | 1 | 2 | UBE2E2 |
| 3 | 172.2 | 4.1 | 1 | 10 | |
| 5 | 32.9 | 4.1 | 1 | 24 | |
| 5 | 99.1 | 3.1 | 1 | 5 | |
| 6 | 7.7 | 1.9 | 1 | 5 | BMP6 |
| 6 | 39.5 | 0.2 | 1 | 1 | KIF6 |
| 6 | 40.7 | 4.4 | 1 | 79 | |
| 6 | 54.1 | 0.8 | 1 | 3 | |
| 7 | 43.5 | 3.8 | 1 | 33 | |
| 7 | 90.1 | 3.5 | 1 | 20 | |
| 7 | 114.7 | 1.4 | 2 | 6 | MET |
| 7 | 116.2 | 0.9 | 1 | 5 | WNT2 |
| 8 | 23.4 | 1.2 | 1 | 6 | ADAM28 |
| 8 | 26.4 | 0.3 | 1 | 2 | |
| 8 | 36.9 | 1.2 | 1 | 11 | ASH2L |
| 8 | 100.3 | 4.4 | 1 | 26 | |
| 9 | 20.1 | 1.3 | 1 | 15 | |
| 9 | 23.2 | 1.7 | 1 | 1 | ELAVL2 |
| 10 | 73.6 | 0.1 | 1 | 2 | |
| 10 | 116.5 | 0.8 | 1 | 3 | |
| 11 | 69.0 | 0.3 | 3 | 5 | CCND1 |
| 11 | 76.1 | 0.4 | 1 | 3 | |
| 11 | 90.7 | 0.2 | 1 | 0 | |
| 11 | 101.3 | 1.8 | 2 | 17 | BIRC2; BIRC3; YAP1 |
| 12 | 18.4 | 0.9 | 1 | 4 | PIK3C2G |
| 12 | 98.0 | 1.6 | 1 | 10 | |
| 13 | 18.3 | 3.6 | 1 | 21 | |
| 13 | 102.2 | 2.4 | 1 | 5 | |
| 13 | 105.8 | 1.0 | 1 | 2 | EFNB2 |
| 14 | 75.8 | 0.9 | 1 | 7 | |
| 15 | 65.4 | 2.6 | 1 | 17 | |
| 16 | 48.7 | 2.4 | 1 | 8 | |
| 19 | 33.3 | 1.6 | 1 | 4 | |
| 20 | 10.2 | 1.0 | 1 | 4 | JAG1 |
| 21 | 41.1 | 0.6 | 1 | 6 | |
| X | 56.0 | 1.8 | 1 | 8 | SPIN2; SPIN3 |

B

| | | | | | |
|---|---|---|---|---|---|
| 2 | 84.8 | 3.6 | 1 | 41 | |
| 4 | 20.1 | 4.1 | 1 | 7 | SLIT2 |
| 4 | 54.3 | 3.9 | 1 | 24 | |
| 4 | 187.8 | 0.2 | 1 | 2 | FAT |
| 6 | 37.9 | 1.6 | 1 | 10 | |
| 6 | 39.7 | 1.0 | 1 | 5 | DAAM2 |
| 8 | 38.2 | 4.9 | 1 | 40 | |
| 9 | 21.0 | 2.8 | 1 | 23 | CDK2NA; CDK2NB |
| 9 | 127.6 | 1.5 | 1 | 46 | |
| 10 | 89.7 | 1.2 | 2 | 7 | PTEN |
| 10 | 117.4 | 4.1 | 1 | 28 | |
| 11 | 46.8 | 1.2 | 1 | 22 | |
| 18 | 53.2 | 2.1 | 1 | 14 | |
| 22 | 40.2 | 1.0 | 1 | 21 | |
| X | 99.5 | 4.7 | 1 | 58 | |

### Example 7

cIAP1 is Consistently Overexpressed in Tumors Harboring the Murine 9qA1 and Human 11q22 Amplicons

One criterion for establishing whether a gene in an amplicon might contribute to tumorigenesis is that it is overexpressed in tumors that contain the amplicon. We further hypothesized that this criterion should hold even across species. Thus, we performed a comprehensive gene expression analysis of overlapping genes from the mouse 9qA1 and human 11q22 amplicons. Specifically, we used real time quantitative PCR (RT-Q-PCR) to measure mRNA levels for all genes in these regions.

Amplicon positive mouse HCCs throughout displayed elevated mRNA levels for most of the MMPs except MMP7, with high variability in maximum expression level. In marked contrast, the mRNA levels for MMP1, MMP3, MMP8, MMP12, MMP13, MMP20 and MMP27 were below detection limit in 25 human HCCs, including a tumor with the 11q22 amplicon. However, MMP7 and MMP10 mRNAs were moderately elevated in the 11q22-positive HCC that underwent thorough expression analysis. Therefore, with the possible exception of MMP10, the matrix metalloproteinases were not consistently overexpressed in amplicon-positive murine and human HCCs and probably not responsible for the selective advantage conferred by this genomic amplification. Furthermore, ROMA analysis on various 11q22 positive human carcinomas identified an ovarian carcinoma harboring an 11q22 amplicon that decisively excluded all of the MMPs. Concordantly, a recent study using low resolution technologies excluded at least some MMPs from an 11q22 amplicon in lung cancer (Dai et al., supra).

Turning towards the other candidate genes in the amplicon, we found that amplicon positive murine HCCs consistently overexpressed cIAP1 and cIAP2 mRNA, and cIAP1 protein. The cIAP1 and cIAP2 genes were overexpressed in murine HCC . tumors containing elevated cIAP1 gene copy number, as determined by quantitative real-time RT-PCR analysis. The cIAP1 protein was also overexpressed in outgrown murine HCC tumor cells containing the 9qA1 amplicon as assayed by immunoblotting using a monoclonal anti-cIAP antibody or a polyclonal anti-cIAP1/2 antiserum. These genes were not upregulated in tumors without the amplicon. Both genes were overexpressed in the human HCC and esophageal tumors harboring the 11q22 amplicon, but also in a substantial number of tumors without cIAP1/cIAP2 copy number elevation (4 of 25 HCC; 15 of 50 esophageal). Interestingly, cIAP1 was the only cIAP overexpressed in human HCCs without 11q22 amplification. Results also demonstrated that cIAP1 promoted the turnover of cIAP2 in a dose-dependent manner in vitro, and showed that cIAP2 protein increased in 9qA1 positive murine HCC cells grown in the presence of a proteasome inhibitor.

Yap mRNA was elevated in all mouse and human amplicon-containing tumors examined. cIAP1 and Yap were consistently overexpressed in mouse and human tumors containing the 9qA1 or 11q22 amplicon, as compared to cIAP1, cIAP2, Yap and Porimin mRNA levels, as determined by RT-Q-PCR analysis in murine and human HCCs. Yap protein was also elevated in protein lysates from 9qA1 positive or negative liver cancers and adult mouse livers, immunoblotted with antibodies against cIAP1, cIAP1/2, YAP and Porimin. Similarly, Porimin mRNA was elevated in all amplicon-containing tumors, although there was no overexpression of the protein in 9qA1 positive mouse tumors or an 11q22 positive human tumor. Based on these aggregate analyses, cIAP1 and Yap are oncogenes.

### Example 8

cIAP1 has Oncogenic Properties

Inhibitor of apoptosis (IAP) proteins were originally identified in baculovirus because of their potential to inhibit cell death of infected cells (Crook et al., “An apoptosis-inhibiting baculovirus gene with a zinc finger-like motif,” J. Tirol. 67, 2168-2174 (1993)). Similar to their viral counterparts, overexpression of cellular IAPs can inhibit apoptosis induced by different stimuli (Liston et al., “Suppression of apoptosis in mammalian cells by NAIP and a related family of IAP genes,” Nature 379, 349-353 (1996); Duckett et al., “A conserved family of cellular genes related to the baculovirus iap gene and encoding apoptosis inhibitors,” EMBO J. 15, 2685-2694 (1996)). Although IAPs can bind and inhibit caspases, it is controversial as to whether they are important regulators of apoptosis in mammalian cells (Liston et al., “The inhibitors of apoptosis: there is more to life than Bc12,” Oncogene 22, 8568-8580 (2003). Furthermore, although indirect evidence point towards a role for IAPB in oncogenesis (Wright and Duckett, “Reawakening the cellular death program in neoplasia through the therapeutic blockade of IAP function,” J. Clin. Invest 115, 2673-2678 (2005)), there is as yet no direct evidence that these genes actively contribute to tumor initiation or maintenance.

A significant advantage of profiling the genomes of defined murine tumors is that candidate genes can be validated in a genetic context in which the mutation spontaneously arose during tumorigenesis. Our studies identified the 9qA1 amplicon in tumors derived from p53−/−hepatoblasts expressing Myc but not in other configurations, indicating that these cells are ideal for evaluating the oncogenic properties of cIAP1. Therefore, p53−/−; myc liver progenitor cells expressing cIAP1 or a control vector were produced using retroviral mediated gene transfer. The resulting cell populations were examined for transgene expression and subjected to different apoptotic triggers. Expression of myc-tagged-cIAP1 in p53−/−; myc liver progenitor cells was confirmed by western blot analysis using a monoclonal anti-cIAP1 antibody. In this cell type, cIAP1 overexpression conferred a modest protection from growth factor withdrawal and spontaneous cell death at confluence. cIAP1 overexpression in p53−/−; myc hepatoblasts suppressed p53 independent forms of apoptosis induced by different death stimuli. Expression of myc-tagged-cIAP1 in p53−/−; myc liver progenitor cells was confirmed by western blot analysis using a monoclonal anti-cIAP1 antibody. p53-I- hepatoblasts, double infected with myc+cIAP1 or myc+vector were grown in decreasing serum concentrations for 48 hrs. cIAP1 expression was also shown to protect hepatoblasts from apoptosis mediated by serum withdrawal.

cIAP1 also protected against spontaneous cell death mediated by contact inhibition, when p53−/−hepatoblasts (myc+cIAP1 or myc+vector) were grown to confluence and apoptosis was measured 24 hours later.

Surprisingly, cIAP1 had no effect on apoptosis induced by the death ligands TRAIL and TNFα, although it did confer substantial short and long-term protection from Fas-mediated apoptosis. p53−/−hepatoblasts (myc+cIAP1 or myc+vector) were treated with 125 ng/ml TRAIL, 5 ng/ml TNFα or increasing concentrations of FasL (25, 50, 100 ng/ml) together with 2.5 ug/ml cycloheximide for 12 hrs and apoptosis was measured. cIAP1 increased short and long-term viability following Fas Ligand (FasL) treatment in p53−/−hepatoblasts (Myc+cIAP1 or Myc+vector) treated with 50 ng/ml FasL for 36 hrs. Separately, p53−/−hepatoblasts (Myc+cIAP1 or Myc+vector) were treated with 50 ng/ml FasL for 36 hrs. cIAP1 increased short and long-term viability following FasL treatment. Taken together, these results demonstrate that cIAP1 protected against FasL triggered cell death but not TNFa or TRAIL mediated cell death of liver progenitor cells. Thus, cIAP1 suppresses apoptosis in murine hepatoblasts in vitro.

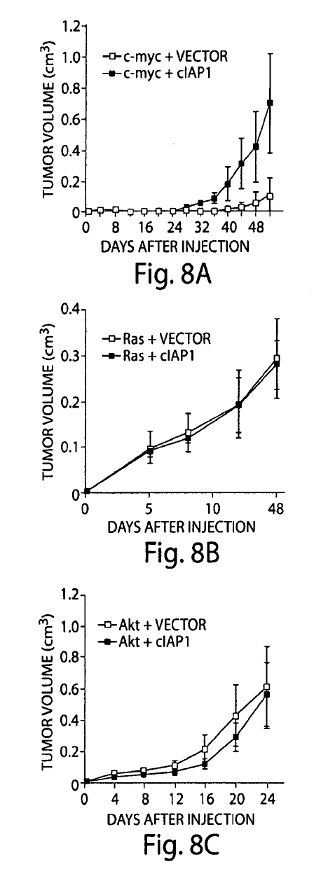

To determine whether cIAP1 could function as an oncogene in vivo, the hepatoblast cultures described above were injected subcutaneously into nude mice to facilitate precise measurement of tumor growth. cIAP1 overexpression significantly accelerated the growth of p53−/−hepatoblasts expressing Myc (FIG. 8A), reducing onset times by half (onset time of 24 ±2.3 days for myc+cIAP1 vs. 45±12.2 days for myc+vector (p=0.02)), and greatly increasing tumor burden. The tumors displayed the histopathology of moderately well to poorly differentiated HCC and stably overexpressed the cIAP1 protein at high levels. Also present were low molecular weight forms of cIAP1, consistent with the susceptibility of this protein to proteolytic degradation. Interestingly, one control tumor that was harvested at a very small size showed elevated levels of cIAP1, suggesting that a subset of these cells had acquired a spontaneous alteration that upregulated the gene.

The ability of cIAP1 to promote tumorigenicity in cooperation with Akt or Ras was also examined. Using the same procedures described above, we produced p53-1- hepatoblasts expressing either Akt or Ras with or without cIAP1. In contrast to the Myc configuration, overexpression of cIAP1 had no impact on the onset or progression of tumors expressing Akt or Ras (FIGS. 8B and C), even though cIAP1 was efficiently expressed. Thus, cIAP1 is selectively oncogenic in the genetic context where its amplification occurs.

### Example 9

cIAP1 and cIAP2 are Required for Rapid Tumor Growth

The above data demonstrate that cIAP1 can causally contribute to HCC development. To determine whether the cIAP proteins were required to sustain tumor growth, the impact of reducing cIAP levels on the growth of Myc-induced HCCs was examined in vivo. We chose to suppress the expression of cIAP1 and cIAP2, since cIAP2 could be upregulated in response to cIAP1 suppression (Conze et al., supra). First, we generated a series of retroviral vectors expressing shRNAs capable of suppressing cIAP1 (hygromycin selectable) and cIAP2 (puromycin selectable) expression by RNA interference. The best performing shRNAs were co-introduced into outgrown Myc-induced HCC cells containing or lacking the 9qA1 amplicon. Using these vectors, we found significant downregulation of endogenous cIAP1/2, as shown by immunoblotting using an antibody directed against cIAP1 or an antibody that cross-reacted with both cIAP1 and cIAP2. Some of these cells were also subsequently injected subcutaneously into the flanks of immunocompromised mice, and tumor growth was assessed by caliper measurement.

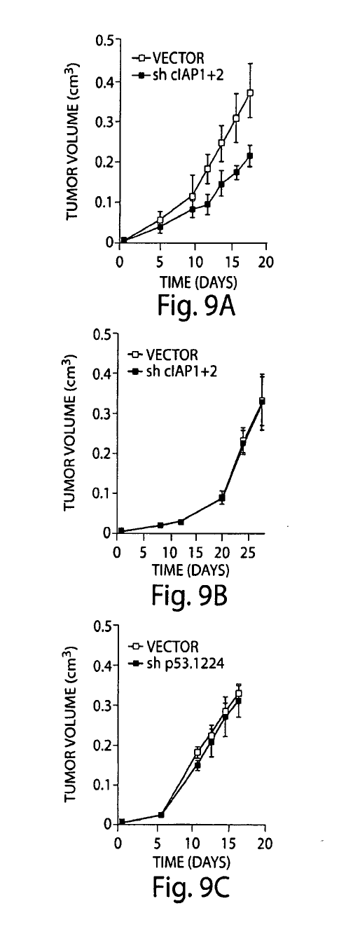

In tumors harboring the 9qA1 amplicon, suppression of cIAP1/2 had a marked impact on tumor growth. Tumors bearing the 9qA1 amplicon show delayed growth upon cIAP1 and cIAP2 suppression. Hepatoma cells outgrown from a 9qA1 amplicon positive, p53−/−; Myc tumor, were double infected with shRNAs targeting cIAP1 and cIAP2 or control vectors (V), or no vector (-). Expression of cIAP1 and cIAP2 was significantly reduced as shown on the immunoblots that were probed with a monoclonal anti-cIAP1 antibody and a polyclonal anti-cIAP1/2 antibody. The levels of XIAP were not reduced. Thus, tumors expressing cIAP1 and cIAP2 shRNAs showed a reduced growth rate compared to parallel tumors expressing the control vectors (FIG. 9A). The efficiency of cIAP knockdown was greatly reduced in the outgrown tumors compared to the injected cells, implying that cells retaining high cIAP levels were selected during tumor expansion. These same shRNAs had no impact on the growth of an amplicon negative tumor derived from the same genotype (FIG. 9B), suggesting that only cells selected for cIAP overexpression were sensitive to cIAP inhibition. This latter observation also rules out off-target effects of these shRNAs on tumor growth. Accordingly, a p53 shRNA did not inhibit the growth of the p53−/−; myc tumor containing the 9qA1 amplicon (FIG. 9C). Therefore, cIAP1 and 2 are required for the efficient growth of tumors harboring the 9qA1 amplicon and thus may be therapeutic targets in a subset of human cancers.

### Example 10

### Yap has Oncogenic Properties and Contributes to Rapid Tumor Growth

In addition to cIAP1, Yap was also overexpressed at the RNA and protein levels in every tumor harboring the mouse 9qA1 or human 11q22 amplicon. Yap (synonyms Yap65 or Yap1) was originally identified due to its interaction with the Src family kinase Yes (Sudol, “Yes-associated protein (YAP65) is a proline-rich phosphoprotein that binds to the SH3 domain of the Yes proto-oncogene product,” Oncogene 9, 2145-2152 (1994)), and acts as a transcriptional co-activator that can bind and activate Runx transcription factors (Yagi et al., “A WW domain-containing yes-associated protein (YAP) is a novel transcriptional co-activator,” EMBO J. 18, 2551-2562 (1999)) as well as the TEAD/TEF transcription factors. In an apparent contradiction to its candidacy as an oncogene, mammalian Yap also interacts with the p53 family member p73 (Strano et al., “Physical interaction with Yes-associated protein enhances p73 transcriptional activity,” J. Biol. Chem. 276, 15164-15173.2001(2001)) and potentiates apoptosis in a manner that is negatively regulated by Akt (Basu et al., “Akt phosphorylates the Yes-associated protein, YAP, to induce interaction with 14-3-3 and attenuation of p73-mediated apoptosis,” Mol. Cell 11, 11-23 (2003)). However, recent studies suggest that Yorkie, the Drosophila homolog of Yap, promotes tissue expansion as an effector of the Lats/Warts pathway by simultaneously activating cyclin E and the Drosophila inhibitor of apoptosis gene dIAP (Huang et al., “The Hippo signaling pathway coordinately regulates cell proliferation and apoptosis by inactivating Yorkie, the Drosophila Homolog of YAP,” Cell 122, 421-434 (2005)). Interestingly, we discovered that murine tumors harboring the 9qA1 amplicon overexpressed cyclin E.

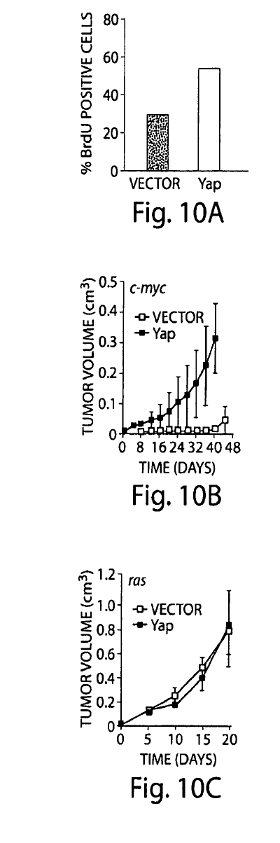

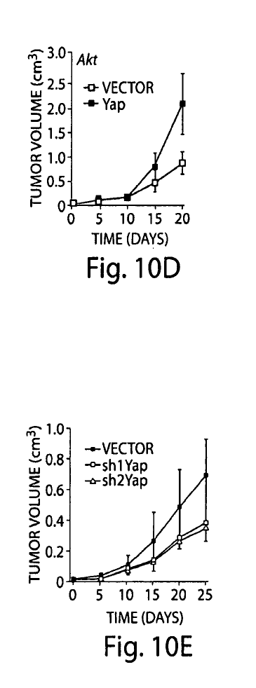

To determine whether Yap could also contribute to the transformation of liver progenitor cells, we conducted functional studies that paralleled our analysis of cIAP1. Consistent with a potential role for Yap in promoting proliferation, we observed that p53−/−cells co-expressing Myc and Yap grew more rapidly than cells expressing Myc alone and displayed a higher BrdU incorporation rate (FIG. 10A).

Furthermore, Yap significantly accelerated tumor onset and progression of myc; p53−/−liver progenitor cells following injection of these cells into immunocompromised mice (FIG. 10B), greatly increasing tumor burden (“myc; vector” vs. “myc; Yap” at day 40, p<0.005). In contrast, Yap did not accelerate tumorigenesis together with activated Ras, although it did enhance Akt driven tumorigenesis, particularly at later time points (FIG. 10C, D).

We also tested whether Yap was required for efficient tumor growth. We generated two Yap-specific shRNAs and showed that each were capable of suppressing Yap. Consistent with cyclin E being one downstream target, cells expressing either Yap shRNA also had reduced cyclin E levels. Despite the incomplete suppression of Yap, cells harboring the 9qA1 amplicon and expressing either Yap shRNA showed slower tumor progression compared to controls following injection into recipient mice (FIG. 10E, p<0.05 [0.013 (shYap 2)/0.018 (shYap 1)] at day 25 post-injection). Together, these data validated Yap as a potent oncogene.

### Example 11

cIAP1 and Yap Cooperate to Promote Tumorigenesis

The data described above identify cIAP1 and Yap as bona fide oncogenes in human cancers such as liver cancer. Whereas cIAP1 may exert its oncogenic potential by suppression of programmed cell death, our data are consistent with a role for Yap in proliferation. The prevailing view in cancer genomics is that focal genomic amplifications contain a key “driver” gene that is selected for during tumorigenesis. However, having validated two oncogenes in one focal amplicon, we next investigated whether cIAP1 and Yap could cooperate during tumorigenesis. “p53−/−; myc” liver progenitor cells were either infected with Yap and control vector or Yap plus cIAP1 and implanted subcutaneously into the flanks of immunocompromised mice, and the recipient animals were monitored for tumor formation using caliper measurements and fluorescence imaging for a co-expressed GFP reporter.

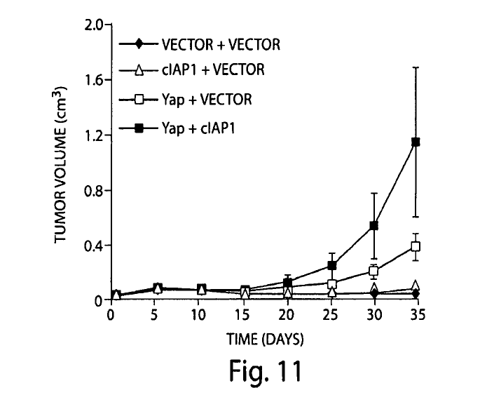

Remarkably, cIAP1 and Yap had a synergistic effect on enhancing tumorigenesis in the context in which their amplification occurred. Thus, tumors arising from “p53−/−; myc” hepatoblasts also co-expressing cIAP1 and Yap formed tumors sooner and grew faster than those expressing either oncogene alone (FIG. 19A; p<0.005 and p<0.05 [0.011] for “cIAP1+Yap” vs. cIAP1 or Yap alone, respectively). These effects were not merely additive: at time points when tumors expressing Yap alone were still small and those harboring cIAP1 alone were as yet barely detectable, tumors co-expressing Yap and cIAP1 were sufficiently large that the animals had to be sacrificed (FIG. 11). Other gene combinations did not have these effects. For example, co-expression of cIAP2 and cIAP1 had no further impact on promoting tumorigenesis compared to cIAP1 alone, and the combination of Porimin with Yap appeared to even delay tumorigenesis. Thus our study establishes for the first time that two adjacent genes from the same focal amplification can cooperate during tumorigenesis.

### Experimental Procedures

### Generation of Genetically Defined Liver Carcinomas, Tumor Re-Transplantation, Analysis, and Immunohistochemistry

All retroviruses were based on MSCV vectors containing human cDNAs encoding for Myc, H-RasV12, Akt, cIAP1 or the murine cDNAs encoding Yap and myc-tagged cIAP1. Short hairpin RNAs against cIAP1, cIAP2 or Yap were expressed from the LTR promoter of MSCV retroviruses. Tumor volume (cm³) was calculated as length x width x height. Paraffin embedded liver tumor sections were stained with Hematoxilin/Eosin according to standard protocols or with α-GFP (Abeam 290). Standard Proteinase K antigen-retrieval was used. Human hepatocellular carcinomas were analyzed using antibodies against: Ck8 (RDI), cIAP1 (Silke et al., “Determination of cell survival by RING-mediated regulation of inhibitor of apoptosis (IAP) protein abundance,” Proc. Natl. Acad. Sci. U.S.A. 102, 16182-16187 (2005)), cIAP2 (sc-7944, Santa Cruz), YAP1 (sc-15407, Santa Cruz), Porimin (IMG472, IMGENEX).

### Immunoblotting

Fresh tumor tissue or cell pellets were lysed in RIPA buffer using a tissue homogenizer. Equal amounts of protein (16 μg) were separated on 10%-SDS-polyacrylamide gels, and transferred to PVDF membranes. The blots were probed with antibodies against cIAP1 (Silke et al., supra), cIAP1/2 (1:2000, gift from P. Liston), YAP1 (sc-15407, Santa Cruz, 1:200), Porimin (IMIG472, IMGENEX, 1:300), Cyclin E (#06-459, Upstate, 1:500), Tubulin (B-5-1-2, Sigma, 1:5000). Vimentin (Abeam, 1:1000), Cytokeratin 19 (Biocare Medical, 1:1000) Albumin (Biogenesis, 1:5000 or AFP (Dako, 1:1000).

### Cell Proliferation Assay and Cell Death ELISA

Cells plated on gelatin coated coverslips were incubated with 5-Bromo-2′-deoxyuridine (BrdU, 100 ug/ml, Sigma) for 1 hr. Nuclei incorporating BrdU were visualized by immunolabeling using anti-BrdU antibody (Pharmingen, 1:400) as previously described (Narita et al., 2003). DNA was visualized by DAPI (1 μg/ml) after permeabilization with 0.2% Triton X-100/PBS. Cells were grown in various concentrations of serum and apoptosis was measured using the Cell Death Detection ELISAPLUS kit (Roche).

### Representational Oligonucleotide Microarray Analysis

Human tumor samples were obtained from the NCI-sponsored Cooperative Human Tissue Network or the tissue bank of the University of Hong Kong, China. Genomic DNA was isolated from human or mouse tumors using the PureGene DNA isolation kit (Gentra). Hybridizations were carried out on 85K arrays (Nimblegen) {(Lucito et al., 2003) (Lakshmi et al., submitted). The genome position was determined from the UCSC GoldenPath browser; freezes April 2003 for human and February 2003 for mouse. Focal gains or losses were defined as spanning <5 MB.

### Quantitative Real-Time PCR

Quantitative real-time PCR was performed on a PRISM 7700 sequence detector (Applied Biosystems, Foster City, Calif.). Quantification of genomic copy number is based on standard curves derived from serial dilutions of normal human genomic DNA (Invitrogen). For quantitation of mRNA expression, mouse tumors were freshly homogenized in Trizol (Gibco) RNA was isolated and treated with RNase free DNase (Qiagen) and purified over Qiagen RNAeasy columns. Total RNA was converted to cDNA using TaqMan reverse transcription reagents (Applied Biosystems) and used in Q-PCR reactions with incorporation of Sybr green PCR Master Mix (Applied Biosystems) done in triplicate using gene specific primers. Quantification of mRNA expression of human tumor samples was performed using Taqman (Applied Biosystems) probes. Samples were normalized to the level of [β-actin.

### Generation, Characterization and Transduction of Hepatoblast Cultures

E-Cad+hepatoblasts were isolated and grown on NIH-3T3 feeder layers. Phase contrast micrograph showed that islands of hepatoblasts attached in close proximity to NIH-3T3 feeder cells. Liver progenitor cells were expanded in culture when grown in chemically defined hepatocyte growth medium. Liver progenitor cells were efficiently infected with GFP-tagged retroviral vectors, as shown by GFP fluorescence.

Characterization of Markers from Myc Driven Murine HCCs

Total protein lysates from six representative p53−/−; myc murine liver tumors were immunoblotted with antibodies against liver/liver tumor markers: Alpha-fetoprotein (AFP), Vimentin, Cytokeratin 19 (CK19) and Albumin. Protein lysates from adult C57/B6 mouse liver and purified embryonic liver progenitor cells, hepatoblasts (LPC), are loaded for comparison. Tumors derived from p53+/−liver progenitor transduced with Akt or myc oncogenes were transplanted into host mice to allow tumor formation. Genomic DNA from cells prior to transplantation and from tumors was analyzed by PCR using p53 allele specific primers (Schmitt et al., supra). The wild-type p53 allele was found to be lost in the tumors.

Characterization of cIAP2

293T cells were transfected with the indicated vectors expressing cIAP1 or FLAG-tagged cIAP2 at varying ratios. Cells were then treated with β-Lactacystin or left untreated. cIAP2 was detectable when cIAP1 was co-transfected at a 10 fold less ratio but was not detectable when cIAP1 was transfected at an equimolar ratio with cIAP2. cIAP2 was found to be ubiquitylated and degraded in a cIAP1 dependent manner. However, the cIAP1-dependent degradation of cIAP2 was prevented by the proteasome inhibitor and there were polyubiquitylated forms of cIAP2 present. Similarly, murine hepatoma cells derived from a 9qA1 amplicon positive HCC (A+) were cultured with or without the proteasome inhibitor β-Lactacystin for 7 hours (25 μM). Whole protein lysates of these cells were immunoblotted with a polyclonal antibody that recognized both cIAP1 and cIAP2. cIAP2 protein was found to be stabilized in the presence of the proteasome inhibitor.

We claim,:

1. A method of diagnosing liver cancer in a human patient, comprising:
providing a DNA sample from the liver of the patient, and
detecting, in said DNA sample, amplification of a nucleic acid sequence in chromosomal region 11q22, wherein said amplification indicates that the patient has, or is susceptible of developing, liver cancer.

2. The method of embodiment 1, wherein the nucleic acid sequence is from a cIAP1, cIAP2 or Yap gene.

3. A method of selecting a cancer patient for treatment with an inhibitor of CDK, RAF, or MEK, comprising:
providing a DNA sample from the cancer tissue of the patient, and
detecting, in said DNA sample, amplification of a nucleic acid sequence in chromosomal region 11q22, wherein said amplification indicates that the patient will be responsive to said treatment.

4. The method of claim 3, wherein the nucleic acid sequence is from a cIAP1, cIAP2 or Yap gene.

5. A method of selecting a liver cancer patient for treatment with an IAP inhibitor, comprising:
providing a DNA sample from the liver cancer tissue of the patient, and
detecting, in said DNA sample, amplification of a nucleic acid sequence in chromosomal region 11q22, wherein said amplification indicates that the patient will be responsive to said treatment.

6. The method of claim 5, wherein the nucleic acid sequence is from a cIAP1, cIAP2 or Yap gene.

7. A method of inhibiting the growth of a cancer cell, comprising contacting the cell with an interfering RNA that inhibits the expression of Yap.

8. The method of claim 7, wherein the cancer cell is a liver cancer cell.

9. The method of claim 7, wherein the cancer cell is an epithelial cancer cell.

10. The method of claim 7, wherein the cancer cell is a human cancer cell.

11. A method of identifying a molecule for treating cancer, comprising:
providing a sample comprising Yap,
contacting said sample with a candidate molecule, and
detecting, in said sample, a decrease in the activity of said Yap, wherein said decrease indicates that the candidate molecule is useful for treating cancer.

12. A mouse at least some of whose cells comprise a genome comprising a heterologous nucleic acid sequence comprising a Yap-coding sequence and an expression control sequence linked operatively thereto, wherein the mouse has cancer, or is more susceptible of developing cancer as compared to a control mouse not having said heterologous nucleic acid sequence.

13. The mouse of claim 12, wherein the mouse is a transgenic mouse.

14. The mouse of claim 12, wherein the mouse is a chimeric mouse some or all of whose hepatocytes comprise said genome.

15. The mouse of claim 12, further comprising a second heterologous nucleic acid sequence comprising a cIAP1-coding sequence and a second expression control sequence linked operatively thereto.

16. The mouse of claim 12, wherein the cells comprising said heterologous nucleic acid sequence further comprise a null mutation of a tumor suppressor gene.

17. The mouse of claim 16, wherein the tumor suppressor gene is p53.

18. The mouse of claim 12, wherein the cells comprising said heterologous nucleic acid sequence further comprise a shRNA against a tumor suppressor gene.

19. The mouse of claim 18, wherein the tumor suppressor gene is p16 or p19.

20. A method of identifying a molecule useful for treating cancer, comprising:
providing the mouse of claim 12, wherein the mouse has developed cancer, and
treating the mouse with a candidate molecule, wherein inhibition of the growth of said cancer indicates the candidate molecule is useful for treating cancer.

21. A mammalian cell comprising (1) a first heterologous nucleic acid sequence comprising a Yap-coding sequence and a first expression control sequence linked operatively thereto, and (2) a second heterologous nucleic acid sequence comprising a cLAP1-coding sequence and a second expression control sequence linked operatively thereto.

22. A method of identifying a molecule useful for treating cancer, comprising:
providing the cell of claim 21, and
contacting the cell with a candidate molecule, wherein inhibition of the growth of the cell indicates the candidate molecule is useful for treating cancer.

23. A method of inhibiting the growth of a cancer cell, comprising contacting the cell with a first interfering RNA that inhibits the expression of Yap and an inhibitor that inhibits the activity of cIAP1.

24. The method of claim 23, wherein the inhibitor is an interfering RNA that inhibits the expression of cIAP1.

25. The method of claim 23, wherein the inhibitor is a SMAC homolog.

26. The method of claim 23, wherein the cancer cell is a human cancer cell.

27. A method of treating cancer, comprising administering to a cancer patient an inhibitor of cIAP1 or cIAP2 and an inhibitor of MEK or CDK.

28. A method of treating cancer, comprising administering to a cancer patient a shRNA against Yap and an inhibitor of MEK or CDK.

29. A method of treating cancer, comprising administering to a cancer patient an inhibitor of cIAP1 and a shRNA against Yap.

30. A method of treating cancer, comprising administering to a cancer patient (1) an inhibitor of cIAP1 or CIAP2, or a shRNA against Yap, and (2) a chemotherapy agent.

31. The method of any of claims 27-30, wherein the cancer tissue of said patient comprises amplification of a nucleic acid sequence in chromosomal region 11q22.
